(19) United States
(12) Patent Application Publication
(10) Pub. No.: US 2015/0080263 A1
(43) Pub. Date: Mar. 19, 2015

(54) PRENATAL SCREENING FOR DOWN SYNDROME AND TRISOMY 18

(71) Applicant: Bioscreening and Diagnostics, LLC, Detroit, MI (US)

(72) Inventors: Ray O. Bahado-Singh, Grosse Pointe Shores, MI (US); Kypros Nicolaides, London (GB)

(21) Appl. No.: 14/394,199

(22) PCT Filed: Apr. 12, 2013
(86) PCT No.: PCT/US2013/036441

§ 371 Date: Oct. 13, 2014

Related U.S. Application Data
(60) Provisional application No. 61/623,741, filed on Apr. 13, 2012.

(51) Int. Cl. G01N 33/70; G01N 33/49; G01N 33/50; G01N 33/68

(52) U.S. Cl. CPC G01N 33/70; G01N 33/6806; G01N 33/492; G01N 33/50; G01N 2570/00; G01N 2800/387
USPC 506/12; 436/98; 436/90; 436/501; 435/7.92; 436/129; 436/111; 436/131

(57) ABSTRACT

The present invention is directed to methods for predicting a pregnant woman's risk of carrying a fetus with Down syndrome (Trisomy 21) or Trisomy 18. The methods are based on measuring one or more metabolites obtained from a pregnant woman's bodily fluid, such as blood or urine, and found to be predictive of Trisomy 21 or Trisomy 18.

### FIELD OF THE INVENTION

The present invention generally relates to a method for detecting Down syndrome (Trisomy 21) or Trisomy 18 in a fetus during prenatal screening. The method is based on measuring one or more metabolites obtained from a pregnant woman's bodily fluid, such as blood or urine.

### BACKGROUND OF THE INVENTION

Down syndrome is a common disorder that occurs in approximately in 1 in 800 newborns. The disorder is due to the presence of 47 rather than the 46 chromosomes found in normal individuals. Affected individuals have an extra number 21 chromosome. Down syndrome causes severe mental retardation and is associated with major birth defects, such as defects of the heart.

The incidence of Down syndrome increases significantly with increasing maternal age. Historically, the prenatal detection of Down syndrome has focused on pregnant women at and over the age of 35, at which ages the risks of Down syndrome approach or exceed the risks of diagnostic procedures utilized to detect fetal Down syndrome. Therefore, the standard method of prenatal screening has involved selecting women for diagnostic amniocentesis on the basis of maternal age. Age, however, is an inadequate screening criterion in itself.

Trisomy 18 is the next most common major chromosomal aneuploidy with a frequency of 1 in 7500 newborns. This disorder is characterized by an extra chromosome from the number 18 group. Thus rather than having two chromosomes for the number 18 group (disomy) such individuals have three copies of chromosome number 18. Affected individuals have severe mental retardation, severe failure to thrive and major birth defects e.g. of the brain and heart.

It is now the standard of obstetric care and widely practiced in obstetric medicine to routinely screen pregnant women for the presence of Down syndrome and/or Trisomy 18 in the fetus (Driscoll D A et al. Am J Obstet General 2009; 200: 459.e1-459.e9; ACOG Committee on Practice Bulletins. ACOG practice bulletin #77: Screening for fetal chromosomal abnormalities. Obstet General 2007; 109: 217-27). Current methods of screening involve the measurement of the concentrations of proteins and other substances in maternal blood (e.g. estriol, human chorionic gonadotrophin (hCG), alpha fetoprotein (AFP) and others). In addition, ultrasound measurement of the amount of fluid at the back of the fetal neck (nuchal translucency) between 11⁺⁰ and 13⁺⁶ weeks has been found to be the strongest single predictor of Down syndrome and Trisomy 18 in the fetus.

Based on the above established facts, a combination of nuchal translucency (NT) measurements, different biochemical marker concentrations in maternal blood and maternal age is routinely recommended and utilized for prenatal screening. For pregnancies found to be at elevated risk for Trisomy 21 or 18 based on such screening, invasive testing, such as amniocentesis or chorionic villus sampling (CVS) is recommended in order to obtain fetal cells for definitive analysis.

### SUMMARY OF THE INVENTION

It is one object of the present invention to provide a method for determining a pregnant woman's risk of carrying a fetus with Down syndrome by measuring concentrations of one or more metabolites selected from the group consisting of 2-hydroxy-butyrate, 3-hydroxy-isovalerate, acetamide, arginine, choline, glycerol, glycine, propylene glycol, carnitine, creatinine, phenylalanine and leucine in the pregnant woman's bodily fluid; comparing the pregnant woman's one or more metabolite concentrations to concentrations of corresponding one or more metabolites obtained from pregnant women carrying Down syndrome fetuses and to concentrations of corresponding one or more metabolites obtained from pregnant women carrying chromosomally normal fetuses, wherein all of the metabolite concentrations are measured at same gestational age; and predicting the pregnant woman's risk of carrying a fetus with Down syndrome, wherein a statistically significant change in the concentration of the one or more metabolites between the pregnant woman and the corresponding one or more metabolites from the pregnant women carrying chromosomally normal fetuses indicates a greater probability of carrying a fetus with Down syndrome.

It is another object of the present invention to provide a method for determining a pregnant woman's risk of carrying a fetus with Trisomy 18 measuring concentrations of one or more metabolites selected from the group consisting of 2-hydroxy-butyrate, acetate, choline, citrate, creatinine, ethanol, formate, glycerol, malonate, methanol, pyruvate, succinate, proline, hydroxy-isovalerate, hydroxy-valerate, and 3-hydroxy-butyrate in the pregnant woman's bodily fluid; comparing the pregnant woman's one or more metabolite concentrations to concentrations of corresponding one or more metabolites obtained from pregnant women carrying fetuses with Trisomy 18 and to concentrations of corresponding one or more metabolites obtained from pregnant women carrying chromosomally normal fetuses, wherein all metabolite concentrations are measured at the same gestational age; and predicting the pregnant woman's risk of carrying a fetus with Trisomy 18, wherein a statistically significant change in the concentration of one or more metabolites between the pregnant woman and the corresponding one or more metabolites from the pregnant women carrying chromosomally normal fetuses indicates a greater probability of carrying a fetus with Trisomy 18.

The present invention is also directed to a computer-readable medium having stored thereon an array of normalized metabolite concentration values and a program of instructions executable by a processor to compare a pregnant woman's bodily fluid sample metabolite concentration value to a corresponding normalized metabolite concentration value obtained from pregnant women carrying chromosomally normal fetuses to predict the pregnant woman's risk of carrying a fetus with Down syndrome or a fetus with Trisomy 18.

Other objects and features will be in part apparent and in part pointed out hereinafter.

### BRIEF DESCRIPTION OF THE DRAWINGS

FIG. 1 shows the statistical separation achieved by three principal components (metabolite sets) in distinguishing Down syndrome cases from normal controls.

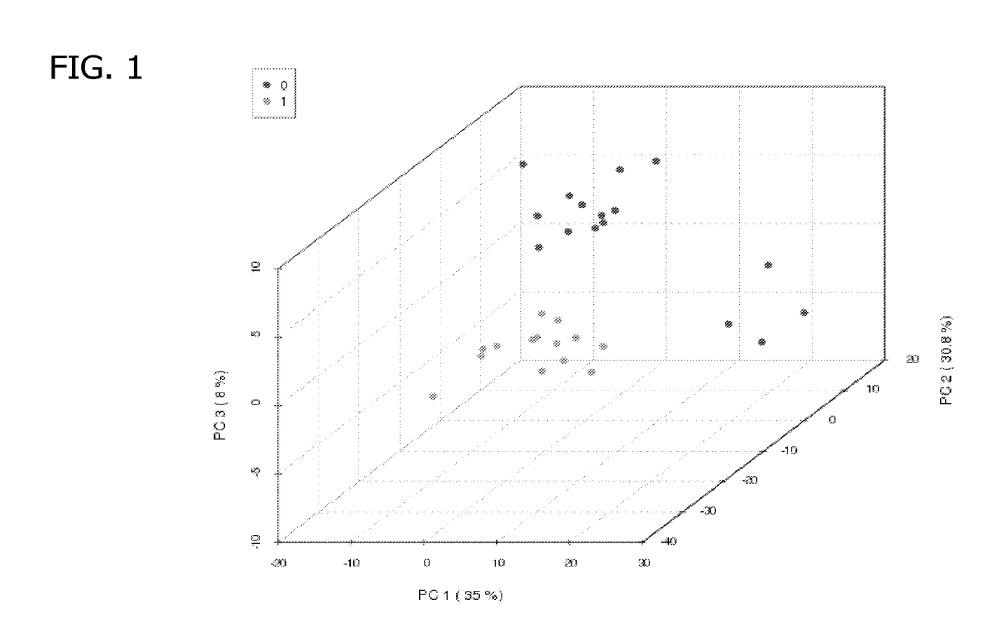

FIG. 2 shows the use of partial least squares discriminant analysis (PLS-DA)—a statistical method which uses a subset of predictor metabolites to achieve maximum separation between the Down syndrome and normal groups. Permutation testing confirms that the separation or discrimination achieved between the two groups i.e. Down syndrome and normal cases is statistically significant, p=0.005.

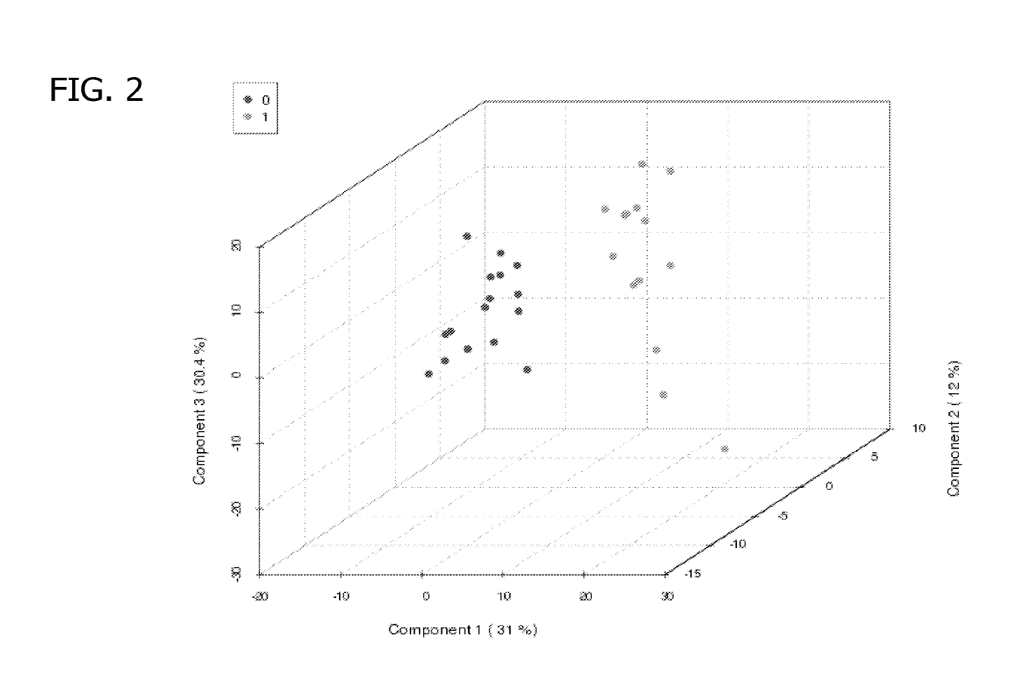

FIG. 3 shows a PLS-DA plot (3D score plot) demonstrating the separation achieved between Trisomy 18 (on green) and normal (in red) cases using Nuclear Magnetic Resonance. Permutation testing confirms that the separation or discrimination achieved between the two groups is statistically significant p=0.029.

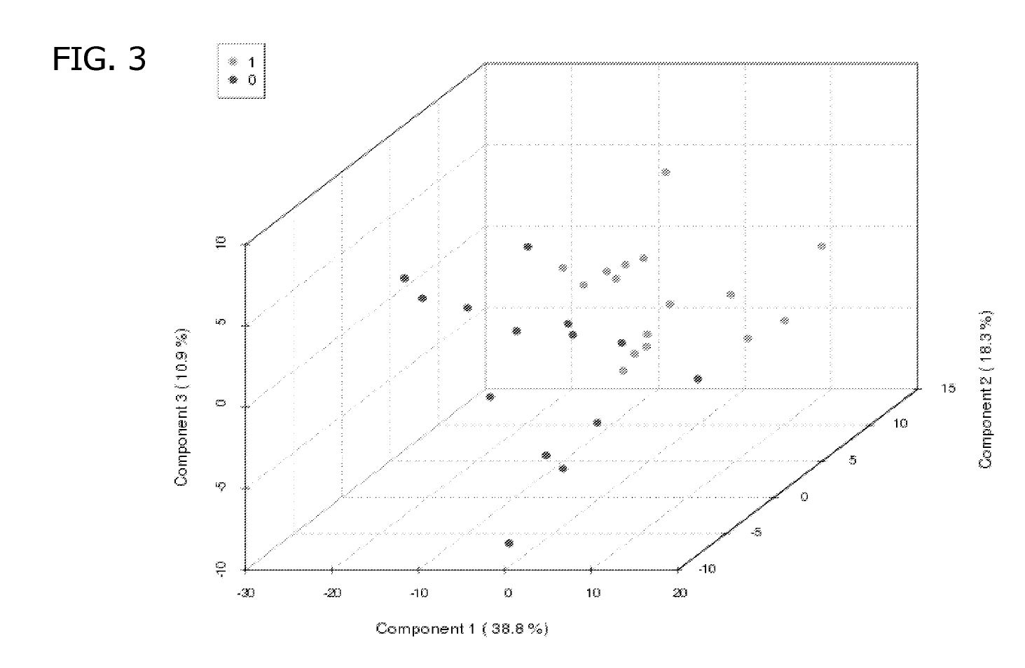

### DEFINITIONS AND ABBREVIATIONS

As used herein, the terms “one or more” and “at least one” in the context of biomarkers, such as metabolites mean any one, two, three, four, etc. of the listed members within a group, in any permutation. Accordingly, the terms “one or more” and “at least one” include any two, any three, any four, etc. of the members specifically listed within a group. Thus, the invention is not limited to any single group or subset of biomarkers. It is emphasized that the terms “one or more” and “at least one” are used in the broadest sense, and are used to designate any subgroup within a group with multiple members. Similarly, the terms “at least 2,” “at least 3,” “at least 4,” etc., cover any combinations of the members within a particular group, provided that the total number of members within the combination is at least 2, at least 3, at least 4, etc.

The term “ionization” and “ionizing” as used herein refers to the process of generating an analyte ion having a net electrical charge equal to one or more electron units. Negative ions are those ions having a net negative charge of one or more electron units, while positive ions are those ions having a net positive charge of one or more electron units.

The term “desorption” as used herein refers to the removal of an analyte from a surface and/or the entry of an analyte into a gaseous phase.

The terms “mass spectrometry” or “MS” as used herein refer to methods of filtering, detecting, and measuring ions based on their mass-to-charge ratio, or “m/z.”

The term “matrix-assisted laser desorption ionization,” or “MALDI” as used herein refers to methods in which a non-volatile sample is exposed to laser irradiation, which desorbs and ionizes analytes in the sample by various ionization pathways, including photo-ionization, protonation, deprotonation, and cluster decay.

“Trisomy 21” and “Down syndrome” are used interchangeably herein.

### DESCRIPTION OF THE PREFERRED EMBODIMENTS

The present invention is directed to methods for determining a pregnant woman's risk of carrying a fetus with Trisomy 21 (Down syndrome) or Trisomy 18. The methods are based on measuring one or more metabolites obtained from a pregnant woman's bodily fluid, such as blood or urine and comparing the concentration of one or more of these metabolites to the corresponding ones isolated from pregnant women carrying chromosomally normal fetuses (i.e., normalized metabolite concentration(s) from normal control pregnant women) and carrying fetuses affected with Trisomy 21 and Trisomy 18 (i.e., normalized metabolite concentration(s) from pregnant women with Trisomy 21 and Trisomy 18).

A method for determining a pregnant woman's risk of carrying a fetus with Down syndrome comprises measuring concentrations of one or more metabolites selected from the group consisting of 2-hydroxy-butyrate, 3-hydroxy-isovalerate, acetamide, arginine, choline, glycerol, glycine, propylene glycol, carnitine, creatinine, phenylalanine and leucine in the pregnant woman's bodily fluid. The pregnant woman's one or more metabolite concentrations are compared to the corresponding one or more metabolite concentrations obtained from pregnant women carrying Down syndrome fetuses and to the corresponding one or more metabolite concentrations obtained from pregnant women carrying chromosomally normal fetuses. All metabolite concentrations are measured at the same or similar gestational age. The pregnant woman's risk of carrying a fetus with Down syndrome is predicted, wherein the statistically significant change in concentration of one or more metabolites between the pregnant woman and the corresponding one or more metabolites from the pregnant women carrying chromosomally normal fetuses indicates a greater probability of carrying a fetus with Down syndrome.

A method for determining a pregnant woman's risk of carrying a fetus with Trisomy 18 comprises measuring concentrations of one or more metabolites selected from the group consisting of 2-hydroxy-butyrate, acetate, choline, citrate, creatinine, ethanol, formate, glycerol, malonate, methanol, pyruvate, succinate, proline, hydroxy-isovalerate, hydroxy-valerate, and 3-hydroxy-butyrate in the pregnant woman's bodily fluid. The pregnant woman's one or more metabolite concentrations are compared to the corresponding one or more metabolite concentrations obtained from pregnant women carrying fetuses with Trisomy 18 and to the corresponding one or more metabolite concentrations obtained from pregnant women carrying chromosomally normal fetuses. All metabolite concentrations are measured at the same or similar gestational age. The pregnant woman's risk of carrying a fetus with Trisomy 18 is predicted, wherein the statistically significant change in concentration of one or more metabolites between the pregnant woman and the corresponding one or more metabolites from the pregnant women carrying chromosomally normal fetuses indicates a greater probability of carrying a fetus with Trisomy 18.

As both methods for predicting a pregnant woman's risk of carrying either a fetus with Down syndrome or Trisomy 18 are quite similar other than for utilizing different metabolites, they are both discussed below in general terms.

Measuring concentrations of one or more metabolites is performed by obtaining the pregnant woman's bodily fluid containing one or more of these metabolites. The bodily fluid can be blood, such as a dried blood sample, a blood serum sample or a blood plasma sample. The bodily fluid can also be urine. Other suitable maternal bodily fluids for use in the methods of the invention include, for example, amniotic fluid, cerebrospinal fluid, mucus, and saliva.

Preferably, a bodily sample such as blood or urine is obtained from a pregnant woman during the first trimester of pregnancy. The blood or urine can be obtained during the second or third trimester. A bodily fluid sample can be obtained from a pregnant woman, for example, at a gestational age from 8 weeks to 18 weeks, from 9 weeks to 14 weeks, or from 10 weeks to 13 weeks.

One or more metabolites used to predict a pregnant woman's risk of carrying a fetus with Trisomy 21 are chosen from a group consisting of 2-hydroxy-butyrate, 3-hydroxy-isovalerate, acetamide, arginine, choline, glycerol, glycine, propylene glycol, carnitine, creatinine, phenylalanine and leucine. In some embodiments, one or more metabolites are selected from the group consisting of propylene glycol, choline, carnitine, acetamide, phenylalanine and 2-hydroxy-butyrate. For example, one or more metabolites are chosen from the group consisting of carnitine, acetamide and 2-hydroxy-butyrate. In other embodiments, one or more metabolites are selected from phenylalanine and choline. Creatinine can be a single metabolite used to test a pregnant woman's bodily fluid, and particularly blood or urine, for risk of carrying a fetus with Trisomy 21. It will be obvious to a skilled artisan that many different combinations of the above-mentioned metabolites for Trisomy 21 can be tested, which include different combinations of 2, 3, 4, 5, 6, 7, 8, 9, 10 or 11 above-mentioned metabolites. Single metabolites and the combination of all 12 metabolites can also be used to test for Trisomy 21 in a fetus.

One or more metabolites used to predict a pregnant woman's risk of carrying a fetus with Trisomy 18 are selected from a group consisting of 2-hydroxy-butyrate, acetate, choline, citrate, creatinine, ethanol, formate, glycerol, malonate, methanol, pyruvate, succinate, proline, hydroxy-isovalerate, hydroxy-valerate, and 3-hydroxy-butyrate. For example, the one or more metabolites used to estimate the risk of carrying a fetus with Trisomy 18 can be selected from glycerol, proline, hydroxy-isovalerate, 3-hydroxy-butyrate, and pyruvate. In other embodiments, the one or more metabolites for Trisomy 18 are selected from glycerol and hydroxyvalerate, or from 2-hydroxy-butyrate, creatinine and ethanol. An example of a single metabolite that can be used to predict a pregnant woman's risk of carrying a fetus with Trisomy 18 is choline. It will be obvious to a skilled artisan that many different combinations of the above-mentioned metabolites for Trisomy 18 can be tested, which include different combinations of 2, 3, 4, 5, 6, 7, 8, 9, 10, 11, 12, 13, 14, or 15 above-mentioned metabolites. Single metabolites and the combination of all 16 metabolites can also be used to test for Trisomy 18 in a fetus.

Representative combinations of 2 metabolites used to predict a pregnant woman's risk of carrying a fetus with Trisomy 21 include:

| 2 Metabolite Combinations | | 2 Metabolite Combinations | |
|---|---|---|---|
| 2-hydroxy-butyrate | 3-hydroxy-isovalerate | 3-hydroxy-isovalerate | Acetamide |
| 2-hydroxy-butyrate | Acetamide | 3-hydroxy-isovalerate | Arginine |
| 2-hydroxy-butyrate | Arginine | 3-hydroxy-isovalerate | Choline |
| 2-hydroxy-butyrate | Choline | 3-hydroxy-isovalerate | Glycerol |
| 2-hydroxy-butyrate | Glycerol | 3-hydroxy-isovalerate | Glycine |
| 2-hydroxy-butyrate | Glycine | 3-hydroxy-isovalerate | Propylene glycol |
| 2-hydroxy-butyrate | Propylene glycol | 3-hydroxy-isovalerate | Carnitine |
| 2-hydroxy-butyrate | Carnitine | 3-hydroxy-isovalerate | creatinine |
| 2-hydroxy-butyrate | creatinine | 3-hydroxy-isovalerate | Phenylalanine |
| 2-hydroxy-butyrate | Phenylalanine | 3-hydroxy-isovalerate | Leucine |
| 2-hydroxy-butyrate | Leucine | Arginine | Leucine |
| Acetamide | Arginine | Arginine | Choline |
| Acetamide | Choline | Arginine | Glycerol |
| Acetamide | Glycerol | Arginine | Glycine |
| Acetamide | Glycine | Arginine | Propylene glycol |
| Acetamide | Propylene glycol | Arginine | Carnitine |
| Acetamide | Carnitine | Arginine | creatinine |
| Acetamide | creatinine | Arginine | Phenylalanine |
| Acetamide | Phenylalanine | Choline | Glycerol |
| Acetamide | Leucine | Choline | Glycine |
| Glycerol | Glycine | Choline | Propylene glycol |
| Glycerol | Propylene glycol | Choline | Carnitine |
| Glycerol | Carnitine | Choline | creatinine |
| Glycerol | creatinine | Choline | Phenylalanine |
| Glycerol | Phenylalanine | Choline | Leucine |
| Glycerol | Leucine | Glycine | Propylene glycol |
| Propylene glycol | Carnitine | Glycine | Carnitine |
| Propylene glycol | creatinine | Glycine | creatinine |
| Propylene glycol | Phenylalanine | Glycine | Phenylalanine |
| Propylene glycol | Leucine | Glycine | Leucine |
| Carnitine | creatinine | Creatinine | Phenylalanine |
| Carnitine | Phenylalanine | Creatinine | Leucine |
| Carnitine | Leucine | Phenylalanine | Leucine |

Representative examples of combinations of 3 metabolites used to predict a pregnant woman's risk of carrying a fetus with Trisomy 21 include:

| 3 Metabolite Combinations | | | 3 Metabolite Combinations | | |
|---|---|---|---|---|---|
| 2-hydroxy-butyrate | 3-hydroxy-isovalerate | Acetamide | 3-hydroxy-isovalerate | Acetamide | Arginine |
| 2-hydroxy-butyrate | 3-hydroxy-isovalerate | Arginine | 3-hydroxy-isovalerate | Acetamide | Choline |
| 2-hydroxy-butyrate | 3-hydroxy-isovalerate | Choline | 3-hydroxy-isovalerate | Acetamide | Glycerol |
| 2-hydroxy-butyrate | 3-hydroxy-isovalerate | Glycerol | 3-hydroxy-isovalerate | Acetamide | Glycine |
| 2-hydroxy-butyrate | 3-hydroxy-isovalerate | Glycine | 3-hydroxy-isovalerate | Acetamide | Propylene |
| | | | | | glycol |
| 2-hydroxy-butyrate | 3-hydroxy-isovalerate | Propylene glycol | 3-hydroxy-isovalerate | Acetamide | Carnitine |
| 2-hydroxy-butyrate | 3-hydroxy-isovalerate | Carnitine | 3-hydroxy-isovalerate | Acetamide | creatinine |
| 2-hydroxy-butyrate | 3-hydroxy-isovalerate | creatinine | 3-hydroxyisovalerate- | Acetamide | Phenylalanine |
| 2-hydroxy-butyrate | 3-hydroxy-isovalerate | Phenylalanine | 3-hydroxy-isovalerate | Acetamide | Leucine |
| 2-hydroxy-butyrate | 3-hydroxy-isovalerate | Leucine | Acetamide | Arginine | Choline |
| Arginine | Choline | Glycerol | Acetamide | Arginine | Glycerol |
| Arginine | Choline | Glycine | Acetamide | Arginine | Glycine |
| Arginine | Choline | Propylene glycol | Acetamide | Arginine | Propylene |
| | | | | | glycol |
| Arginine | Choline | Carnitine | Acetamide | Arginine | Carnitine |
| Arginine | Choline | creatinine | Acetamide | Arginine | creatinine |
| Arginine | Choline | Phenylalanine | Acetamide | Arginine | Phenylalanine |
| Arginine | Choline | Leucine | Acetamide | Arginine | Leucine |
| Choline | Glycerol | Glycine | Glycerol | Glycine | Propylene |
| | | | | | glycol |
| Choline | Glycerol | Propylene glycol | Glycerol | Glycine | Carnitine |
| Choline | Glycerol | Carnitine | Glycerol | Glycine | creatinine |
| Choline | Glycerol | creatinine | Glycerol | Glycine | Phenylalanine |
| Choline | Glycerol | Phenylalanine | Glycerol | Glycine | Leucine |
| Choline | Glycerol | Leucine | Glycine | Propylene | Carnitine |
| | | | | glycol | |
| Propylene glycol | Carnitine | creatinine | Glycine | Propylene | creatinine |
| | | | | glycol | |
| Propylene glycol | Carnitine | Phenylalanine | Glycine | Propylene | Phenylalanine |
| | | | | glycol | |
| Propylene glycol | Carnitine | Leucine | Glycine | Propylene | Leucine |
| | | | | glycol | |
| Carnitine | Creatinine | Phenylalanine | Creatinine | Phenylalanine | Leucine |
| Carnitine | creatinine | Leucine | | | |

Representative combinations of 4 metabolites that can be used to predict a pregnant woman's risk of carrying a fetus with Trisomy 21 include:

4 Metabolite Combinations

| | | | |
|---|---|---|---|
| 2-hydroxy-butyrate | 3-hydroxy-isovalerate | Acetamide | Arginine |
| 2-hydroxy-butyrate | 3-hydroxy-isovalerate | Acetamide | Choline |
| 2-hydroxy-butyrate | 3-hydroxy-isovalerate | Acetamide | Glycerol |
| 2-hydroxy-butyrate | 3-hydroxy-isovalerate | Acetamide | Glycine |
| 2-hydroxy-butyrate | 3-hydroxy-isovalerate | Acetamide | Propylene glycol |
| 2-hydroxy-butyrate | 3-hydroxy-isovalerate | Acetamide | Carnitine |
| 2-hydroxy-butyrate | 3-hydroxy-isovalerate | Acetamide | creatinine |
| 2-hydroxy-butyrate | 3-hydroxy-isovalerate | Acetamide | Phenylalanine |
| 2-hydroxy-butyrate | 3-hydroxy-isovalerate | Acetamide | Leucine |
| 3-hydroxy-isovalerate | Acetamide | Arginine | Choline |
| 3-hydroxy-isovalerate | Acetamide | Arginine | Glycerol |
| 3-hydroxy-isovalerate | Acetamide | Arginine | Glycine |
| 3-hydroxy-isovalerate | Acetamide | Arginine | Propylene glycol |
| 3-hydroxy-isovalerate | Acetamide | Arginine | Carnitine |
| 3-hydroxy-isovalerate | Acetamide | Arginine | creatinine |
| 3-hydroxy-isovalerate | Acetamide | Arginine | Phenylalanine |
| 3-hydroxy-isovalerate | Acetamide | Arginine | Leucine |
| Acetamide | Arginine | Choline | Glycerol |
| Acetamide | Arginine | Choline | Glycine |
| Acetamide | Arginine | Choline | Propylene glycol |
| Acetamide | Arginine | Choline | Carnitine |
| Acetamide | Arginine | Choline | creatinine |
| Acetamide | Arginine | Choline | Phenylalanine |
| Acetamide | Arginine | Choline | Leucine |
| Arginine | Choline | Glycerol | Glycine |
| Arginine | Choline | Glycerol | Propylene glycol |
| Arginine | Choline | Glycerol | Carnitine |
| Arginine | Choline | Glycerol | creatinine |
| Arginine | Choline | Glycerol | Phenylalanine |
| Arginine | Choline | Glycerol | Leucine |
| Choline | Glycerol | Glycine | Propylene glycol |
| Choline | Glycerol | Glycine | Carnitine |
| Choline | Glycerol | Glycine | creatinine |
| Choline | Glycerol | Glycine | Phenylalanine |
| Choline | Glycerol | Glycine | Leucine |
| Glycerol | Glycine | Propylene glycol | Carnitine |
| Glycerol | Glycine | Propylene glycol | creatinine |
| Glycerol | Glycine | Propylene glycol | Phenylalanine |
| Glycerol | Glycine | Propylene glycol | Leucine |
| Glycine | Propylene glycol | Carnitine | creatinine |
| Glycine | Propylene glycol | Carnitine | Phenylalanine |
| Glycine | Propylene glycol | Carnitine | Leucine |
| Propylene glycol | Carnitine | creatinine | Phenylalanine |
| Propylene glycol | Carnitine | creatinine | Leucine |
| Carnitine | creatinine | Phenylalanine | Leucine |

Representative combinations of 5 metabolite combinations used to predict a pregnant woman's risk of carrying a fetus with Trisomy 21 include:

5 Metabolite Combinations

| | | | | |
|---|---|---|---|---|
| 2-hydroxy-butyrate | 3-hydroxy-isovalerate | Acetamide | Arginine | Choline |
| 2-hydroxy-butyrate | 3-hydroxy-isovalerate | Acetamide | Arginine | Glycerol |
| 2-hydroxy-butyrate | 3-hydroxy-isovalerate | Acetamide | Arginine | Glycine |
| 2-hydroxy-butyrate | 3-hydroxy-isovalerate | Acetamide | Arginine | Propylene |
| | | | | glycol |
| 2-hydroxy-butyrate | 3-hydroxy-isovalerate | Acetamide | Arginine | Carnitine |
| 2-hydroxy-butyrate | 3-hydroxy-isovalerate | Acetamide | Arginine | creatinine |
| 2-hydroxy-butyrate | 3-hydroxy-isovalerate | Acetamide | Arginine | Phenylalanine |
| 2-hydroxy-butyrate | 3-hydroxy-isovalerate | Acetamide | Arginine | Leucine |
| 3-hydroxy-isovalerate | Acetamide | Arginine | Choline | Glycerol |
| 3-hydroxy-isovalerate | Acetamide | Arginine | Choline | Glycine |
| 3-hydroxy-isovalerate | Acetamide | Arginine | Choline | Propylene |
| | | | | glycol |
| 3-hydroxy-isovalerate | Acetamide | Arginine | Choline | Carnitine |
| 3-hydroxy-isovalerate | Acetamide | Arginine | Choline | creatinine |
| 3-hydroxy-isovalerate | Acetamide | Arginine | Choline | Phenylalanine |
| 3-hydroxy-isovalerate | Acetamide | Arginine | Choline | Leucine |
| Acetamide | Arginine | Choline | Glycerol | Glycine |
| Acetamide | Arginine | Choline | Glycerol | Propylene |
| | | | | glycol |
| Acetamide | Arginine | Choline | Glycerol | Carnitine |
| Acetamide | Arginine | Choline | Glycerol | creatinine |
| Acetamide | Arginine | Choline | Glycerol | Phenylalanine |
| Acetamide | Arginine | Choline | Glycerol | Leucine |
| Arginine | Choline | Glycerol | Glycine | Propylene |
| | | | | glycol |
| Arginine | Choline | Glycerol | Glycine | Carnitine |
| Arginine | Choline | Glycerol | Glycine | creatinine |
| Arginine | Choline | Glycerol | Glycine | Phenylalanine |
| Arginine | Choline | Glycerol | Glycine | Leucine |
| Choline | Glycerol | Glycine | Propylene | Carnitine |
| | | | glycol | |
| Choline | Glycerol | Glycine | Propylene | creatinine |
| | | | glycol | |
| Choline | Glycerol | Glycine | Propylene | Phenylalanine |
| | | | glycol | |
| Choline | Glycerol | Glycine | Propylene | Leucine |
| | | | glycol | |
| Glycerol | Glycine | Propylene | Carnitine | creatinine |
| | | glycol | | |
| Glycerol | Glycine | Propylene | Carnitine | Phenylalanine |
| | | glycol | | |
| Glycerol | Glycine | Propylene | Carnitine | Leucine |
| | | glycol | | |
| Glycine | Propylene glycol | Carnitine | creatinine | Phenylalanine |
| Glycine | Propylene glycol | Carnitine | creatinine | Leucine |
| Propylene glycol | Carnitine | creatinine | Phenylalanine | Leucine |

Other representative combinations of 6, 7, 8, 9, 10 or 11 metabolites for Trisomy 21 can be readily determined by a skilled artisan using routine experimentation.

Similarly, metabolite combinations for determining a pregnant woman's risk of carrying a fetus with Trisomy 18 are determined in the same fashion as the ones described above for Trisomy 21.

Thus, representative 2 metabolite combinations used to predict a pregnant woman's risk of carrying a fetus with Trisomy 18 include:

| 2 Metabolite Combinations | | 2 Metabolite Combinations | |
|---|---|---|---|
| 2-hydroxy-butyrate | acetate | acetate | choline |
| 2-hydroxy-butyrate | choline | acetate | citrate |
| 2-hydroxy-butyrate | citrate | acetate | creatinine |
| 2-hydroxy-butyrate | creatinine | acetate | ethanol |
| 2-hydroxy-butyrate | ethanol | acetate | formate |
| 2-hydroxy-butyrate | formate | acetate | glycerol |
| 2-hydroxy-butyrate | glycerol | acetate | malonate |
| 2-hydroxy-butyrate | malonate | acetate | methanol |
| 2-hydroxy-butyrate | methanol | acetate | pyruvate |
| 2-hydroxy-butyrate | pyruvate | acetate | succinate |
| 2-hydroxy-butyrate | succinate | acetate | proline |
| 2-hydroxy-butyrate | proline | acetate | hydroxy-isovalerate |
| 2-hydroxy-butyrate | hydroxy-isovalerate | acetate | hydroxy-valerate |
| 2-hydroxy-butyrate | hydroxy-valerate | acetate | 3-hydroxy-butyrate |
| 2-hydroxy-butyrate | 3-hydroxy-butyrate | choline | citrate |
| Citrate | creatinine | choline | creatinine |
| Citrate | ethanol | choline | ethanol |
| Citrate | formate | choline | formate |
| Citrate | glycerol | choline | glycerol |
| Citrate | malonate | choline | malonate |
| Citrate | methanol | choline | methanol |
| Citrate | pyruvate | choline | pyruvate |
| Citrate | succinate | choline | succinate |
| Citrate | proline | choline | proline |
| Citrate | hydroxy-isovalerate | choline | hydroxy-isovalerate |
| Citrate | hydroxy-valerate | choline | hydroxy-valerate |
| Citrate | 3-hydroxy-butyrate | Choline | 3-hydroxy-butyrate |
| creatinine | ethanol | ethanol | formate |
| creatinine | formate | ethanol | glycerol |
| creatinine | glycerol | ethanol | malonate |
| creatinine | malonate | ethanol | methanol |
| creatinine | methanol | ethanol | pyruvate |
| creatinine | pyruvate | ethanol | succinate |
| creatinine | succinate | ethanol | proline |
| creatinine | proline | ethanol | hydroxy-isovalerate |
| creatinine | hydroxy-isovalerate | ethanol | hydroxy-valerate |
| Creatinine | 3-hydroxy-butyrate | Ethanol | 3-hydroxy-butyrate |
| creatinine | hydroxy-valerate | formate | glycerol |
| glycerol | malonate | formate | malonate |
| glycerol | methanol | formate | methanol |
| glycerol | pyruvate | formate | pyruvate |
| glycerol | succinate | formate | succinate |
| glycerol | proline | formate | proline |
| glycerol | hydroxy-isovalerate | formate | hydroxy-isovalerate |
| glycerol | hydroxy-valerate | formate | hydroxy-valerate |
| Glycerol | 3-hydroxy-butyrate | Formate | 3-hydroxy-butyrate |
| malonate | methanol | methanol | pyruvate |
| malonate | pyruvate | methanol | succinate |
| malonate | succinate | methanol | proline |
| malonate | proline | methanol | hydroxy-isovalerate |
| malonate | hydroxy-isovalerate | methanol | hydroxy-valerate |
| Malonate | 3-hydroxy-butyrate | Methanol | 3-hydroxy-butyrate |
| malonate | hydroxy-valerate | pyruvate | succinate |
| succinate | proline | pyruvate | proline |
| succinate | hydroxy-isovalerate | pyruvate | hydroxy-isovalerate |
| succinate | hydroxy-valerate | pyruvate | hydroxy-valerate |
| Succinate | 3-hydroxy-butyrate | Pyruvate | 3-hydroxy-butyrate |
| proline | hydroxy-isovalerate | hydroxy-isovalerate | hydroxy-valerate |
| proline | hydroxy-valerate | Hydroxy-isovalerate | 3-hydroxy-butyrate |
| Proline | 3-hydroxy-butyrate | Hydroxy-valerate | 3-hydroxy-butyrate |

Representative 3 metabolite combinations used to predict a pregnant woman's risk of carrying a fetus with Trisomy 18 include:

| 3 Metabolite Combinations | | | 3 Metabolite Combinations | | |
|---|---|---|---|---|---|
| 2-hydroxy-butyrate | acetate | choline | acetate | choline | citrate |
| 2-hydroxy-butyrate | acetate | citrate | acetate | choline | creatinine |
| 2-hydroxy-butyrate | acetate | creatinine | acetate | choline | ethanol |
| 2-hydroxy-butyrate | acetate | ethanol | acetate | choline | formate |
| 2-hydroxy-butyrate | acetate | formate | acetate | choline | glycerol |
| 2-hydroxy-butyrate | acetate | glycerol | acetate | choline | malonate |
| 2-hydroxy-butyrate | acetate | malonate | acetate | choline | methanol |
| 2-hydroxy-butyrate | acetate | methanol | acetate | choline | pyruvate |
| 2-hydroxy-butyrate | acetate | pyruvate | acetate | choline | succinate |
| 2-hydroxy-butyrate | acetate | succinate | acetate | choline | proline |
| 2-hydroxy-butyrate | acetate | proline | acetate | choline | hydroxy- |
| | | | | | isovalerate |
| 2-hydroxy-butyrate | acetate | hydroxy- | acetate | choline | hydroxy- |
| | | isovalerate | | | valerate |
| 2-hydroxy-butyrate | acetate | hydroxy- | acetate | choline | 3-hydroxy- |
| | | valerate | | | butyrate |
| 2-hydroxy-butyrate | acetate | 3-hydroxy- | choline | creatinine | ethanol |
| | | butyrate | | | |
| creatinine | ethanol | formate | choline | creatinine | formate |
| creatinine | ethanol | glycerol | choline | creatinine | glycerol |
| creatinine | ethanol | malonate | choline | creatinine | malonate |
| creatinine | ethanol | methanol | choline | creatinine | methanol |
| creatinine | ethanol | pyruvate | choline | creatinine | pyruvate |
| creatinine | ethanol | succinate | choline | creatinine | succinate |
| creatinine | ethanol | proline | choline | creatinine | proline |
| creatinine | ethanol | hydroxy- | choline | creatinine | hydroxy- |
| | | isovalerate | | | isovalerate |
| creatinine | ethanol | hydroxy- | choline | creatinine | hydroxy- |
| | | valerate | | | valerate |
| creatinine | ethanol | 3-hydroxy- | choline | creatinine | 3-hydroxy- |
| | | butyrate | | | butyrate |
| ethanol | formate | glycerol | formate | glycerol | malonate |
| ethanol | formate | malonate | formate | glycerol | methanol |
| ethanol | formate | methanol | formate | glycerol | pyruvate |
| ethanol | formate | pyruvate | formate | glycerol | succinate |
| ethanol | formate | succinate | formate | glycerol | proline |
| ethanol | formate | proline | formate | glycerol | hydroxy- |
| | | | | | isovalerate |
| ethanol | formate | hydroxy- | formate | glycerol | hydroxy- |
| | | isovalerate | | | valerate |
| ethanol | formate | hydroxy- | formate | glycerol | 3-hydroxy- |
| | | valerate | | | butyrate |
| ethanol | formate | 3-hydroxy- | glycerol | malonate | methanol |
| | | butyrate | | | |
| malonate | methanol | pyruvate | glycerol | malonate | pyruvate |
| malonate | methanol | succinate | glycerol | malonate | succinate |
| malonate | methanol | proline | glycerol | malonate | proline |
| malonate | methanol | hydroxy- | glycerol | malonate | hydroxy- |
| | | isovalerate | | | isovalerate |
| malonate | methanol | hydroxy- | glycerol | malonate | hydroxy- |
| | | valerate | | | valerate |
| malonate | methanol | 3-hydroxy- | Glycerol | malonate | 3-hydroxy- |
| | | butyrate | | | butyrate |
| methanol | pyruvate | succinate | pyruvate | succinate | proline |
| methanol | pyruvate | proline | pyruvate | succinate | hydroxy- |
| | | | | | isovalerate |
| methanol | pyruvate | hydroxy- | pyruvate | succinate | hydroxy- |
| | | isovalerate | | | valerate |
| methanol | pyruvate | hydroxy- | pyruvate | succinate | 3-hydroxy- |
| | | valerate | | | butyrate |
| methanol | pyruvate | 3-hydroxy- | succinate | proline | hydroxy- |
| | | butyrate | | | isovalerate |
| proline | hydroxy- | hydroxy- | succinate | proline | hydroxy- |
| | isovalerate | valerate | | | valerate |
| proline | hydroxy- | 3-hydroxy- | succinate | proline | 3-hydroxy- |
| | isovalerate | butyrate | | | butyrate |
| hydroxy-isovalerate | hydroxy- | 3-hydroxy- | | | |
| | valerate | butyrate | | | |

Representative 4 metabolite combinations for diagnosing a pregnant woman's risk for carrying a fetus with Trisomy 18 include:

4 Metabolite Combinations

| | | | |
|---|---|---|---|
| 2-hydroxy-butyrate | acetate | choline | citrate |
| 2-hydroxy-butyrate | acetate | choline | creatinine |
| 2-hydroxy-butyrate | acetate | choline | ethanol |
| 2-hydroxy-butyrate | acetate | choline | formate |
| 2-hydroxy-butyrate | acetate | choline | glycerol |
| 2-hydroxy-butyrate | acetate | choline | malonate |
| 2-hydroxy-butyrate | acetate | choline | methanol |
| 2-hydroxy-butyrate | acetate | choline | pyruvate |
| 2-hydroxy-butyrate | acetate | choline | succinate |
| 2-hydroxy-butyrate | acetate | choline | proline |
| 2-hydroxy-butyrate | acetate | choline | hydroxy-isovalerate |
| 2-hydroxy-butyrate | acetate | choline | hydroxy-valerate |
| 2-hydroxy-butyrate | acetate | choline | 3-hydroxy-butyrate |
| acetate | choline | citrate | creatinine |
| acetate | choline | citrate | ethanol |
| acetate | choline | citrate | formate |
| acetate | choline | citrate | glycerol |
| acetate | choline | citrate | malonate |
| acetate | choline | citrate | methanol |
| acetate | choline | citrate | pyruvate |
| acetate | choline | citrate | succinate |
| acetate | choline | citrate | proline |
| acetate | choline | citrate | hydroxy-isovalerate |
| acetate | choline | citrate | hydroxy-valerate |
| acetate | choline | citrate | 3-hydroxy-butyrate |
| choline | citrate | creatinine | ethanol |
| choline | citrate | creatinine | formate |
| choline | citrate | creatinine | glycerol |
| choline | citrate | creatinine | malonate |
| choline | citrate | creatinine | methanol |
| choline | citrate | creatinine | pyruvate |
| choline | citrate | creatinine | succinate |
| choline | citrate | creatinine | proline |
| choline | citrate | creatinine | hydroxy-isovalerate |
| choline | citrate | creatinine | hydroxy-valerate |
| choline | citrate | creatinine | 3-hydroxy-butyrate |
| citrate | creatinine | ethanol | formate |
| citrate | creatinine | ethanol | glycerol |
| citrate | creatinine | ethanol | malonate |
| citrate | creatinine | ethanol | methanol |
| citrate | creatinine | ethanol | pyruvate |
| citrate | creatinine | ethanol | succinate |
| citrate | creatinine | ethanol | proline |
| citrate | creatinine | ethanol | hydroxy-isovalerate |
| citrate | creatinine | ethanol | hydroxy-valerate |
| citrate | creatinine | ethanol | 3-hydroxy-butyrate |
| creatinine | ethanol | formate | glycerol |
| creatinine | ethanol | formate | malonate |
| creatinine | ethanol | formate | methanol |
| creatinine | ethanol | formate | pyruvate |
| creatinine | ethanol | formate | succinate |
| creatinine | ethanol | formate | proline |
| creatinine | ethanol | formate | hydroxy-isovalerate |
| creatinine | ethanol | formate | hydroxy-valerate |
| creatinine | ethanol | formate | 3-hydroxy-butyrate |
| ethanol | formate | glycerol | malonate |
| ethanol | formate | glycerol | methanol |
| ethanol | formate | glycerol | pyruvate |
| ethanol | formate | glycerol | succinate |
| ethanol | formate | glycerol | proline |
| ethanol | formate | glycerol | hydroxy-isovalerate |
| ethanol | formate | glycerol | hydroxy-valerate |
| ethanol | formate | glycerol | 3-hydroxy-butyrate |
| Formate | glycerol | malonate | methanol |
| Formate | glycerol | malonate | pyruvate |
| Formate | glycerol | malonate | succinate |
| Formate | glycerol | malonate | proline |
| Formate | glycerol | malonate | hydroxy-isovalerate |
| Formate | glycerol | malonate | hydroxy-valerate |
| Formate | glycerol | malonate | 3-hydroxy-butyrate |
| glycerol | malonate | methanol | pyruvate |
| glycerol | malonate | methanol | succinate |
| glycerol | malonate | methanol | proline |
| glycerol | malonate | methanol | hydroxy-isovalerate |
| glycerol | malonate | methanol | hydroxy-valerate |
| glycerol | malonate | methanol | 3-hydroxy-butyrate |
| malonate | methanol | pyruvate | succinate |
| malonate | methanol | pyruvate | proline |
| malonate | methanol | pyruvate | hydroxy-isovalerate |
| malonate | methanol | pyruvate | hydroxy-valerate |
| malonate | methanol | pyruvate | 3-hydroxy-butyrate |
| methanol | pyruvate | succinate | proline |
| methanol | pyruvate | succinate | hydroxy-isovalerate |
| methanol | pyruvate | succinate | hydroxy-valerate |
| methanol | pyruvate | succinate | 3-hydroxy-butyrate |
| pyruvate | succinate | proline | hydroxy-isovalerate |
| pyruvate | succinate | proline | hydroxy-valerate |
| pyruvate | succinate | proline | 3-hydroxy-butyrate |
| succinate | proline | hydroxy-isovalerate | hydroxy-valerate |
| succinate | proline | hydroxy-isovalerate | 3-hydroxy-butyrate |
| proline | hydroxy-isovalerate | hydroxy-valerate | 3-hydroxy-butyrate |

Representative combinations of 5 metabolites that can be used to determine a pregnant woman's risk of carrying a fetus with Trisomy 18 include:

5 Metabolite Combinations

| | | | | |
|---|---|---|---|---|
| 2-hydroxy-butyrate | acetate | choline | citrate | creatinine |
| 2-hydroxy-butyrate | acetate | choline | citrate | ethanol |
| 2-hydroxy-butyrate | acetate | choline | citrate | formate |
| 2-hydroxy-butyrate | acetate | choline | citrate | glycerol |
| 2-hydroxy-butyrate | acetate | choline | citrate | malonate |
| 2-hydroxy-butyrate | acetate | choline | citrate | methanol |
| 2-hydroxy-butyrate | acetate | choline | citrate | pyruvate |
| 2-hydroxy-butyrate | acetate | choline | citrate | succinate |
| 2-hydroxy-butyrate | acetate | choline | citrate | proline |
| 2-hydroxy-butyrate | acetate | choline | citrate | hydroxy-isovalerate |
| 2-hydroxy-butyrate | acetate | choline | citrate | hydroxy-valerate |
| 2-hydroxy-butyrate | acetate | choline | citrate | 3-hydroxy-butyrate |
| acetate | choline | citrate | creatinine | ethanol |
| acetate | choline | citrate | creatinine | formate |
| acetate | choline | citrate | creatinine | glycerol |
| acetate | choline | citrate | creatinine | malonate |
| acetate | choline | citrate | creatinine | methanol |
| acetate | choline | citrate | creatinine | pyruvate |
| acetate | choline | citrate | creatinine | succinate |
| acetate | choline | citrate | creatinine | proline |
| acetate | choline | citrate | creatinine | hydroxy-isovalerate |
| acetate | choline | citrate | creatinine | hydroxy-valerate |
| acetate | choline | citrate | creatinine | 3-hydroxy-butyrate |
| choline | citrate | creatinine | ethanol | formate |
| choline | citrate | creatinine | ethanol | glycerol |
| choline | citrate | creatinine | ethanol | malonate |
| choline | citrate | creatinine | ethanol | methanol |
| choline | citrate | creatinine | ethanol | pyruvate |
| choline | citrate | creatinine | ethanol | succinate |
| choline | citrate | creatinine | ethanol | proline |
| choline | citrate | creatinine | ethanol | hydroxy-isovalerate |
| choline | citrate | creatinine | ethanol | hydroxy-valerate |
| choline | citrate | creatinine | ethanol | 3-hydroxy-butyrate |
| citrate | creatinine | ethanol | formate | glycerol |
| citrate | creatinine | ethanol | formate | malonate |
| citrate | creatinine | ethanol | formate | methanol |
| citrate | creatinine | ethanol | formate | pyruvate |
| citrate | creatinine | ethanol | formate | succinate |
| citrate | creatinine | ethanol | formate | proline |
| citrate | creatinine | ethanol | formate | hydroxy-isovalerate |
| citrate | creatinine | ethanol | formate | hydroxy-valerate |
| citrate | creatinine | ethanol | formate | 3-hydroxy-butyrate |
| creatinine | ethanol | formate | glycerol | malonate |
| creatinine | ethanol | formate | glycerol | methanol |
| creatinine | ethanol | formate | glycerol | pyruvate |
| creatinine | ethanol | formate | glycerol | succinate |
| creatinine | ethanol | formate | glycerol | proline |
| creatinine | ethanol | formate | glycerol | hydroxy-isovalerate |
| creatinine | ethanol | formate | glycerol | hydroxy-valerate |
| creatinine | ethanol | formate | glycerol | 3-hydroxy-butyrate |
| ethanol | formate | glycerol | malonate | methanol |
| ethanol | formate | glycerol | malonate | pyruvate |
| ethanol | formate | glycerol | malonate | succinate |
| ethanol | formate | glycerol | malonate | proline |
| ethanol | formate | glycerol | malonate | hydroxy-isovalerate |
| ethanol | formate | glycerol | malonate | hydroxy-valerate |
| ethanol | formate | glycerol | malonate | 3-hydroxy-butyrate |
| Formate | glycerol | malonate | methanol | pyruvate |
| Formate | glycerol | malonate | methanol | succinate |
| Formate | glycerol | malonate | methanol | proline |
| Formate | glycerol | malonate | methanol | hydroxy-isovalerate |
| Formate | glycerol | malonate | methanol | hydroxy-valerate |
| Formate | glycerol | malonate | methanol | 3-hydroxy-butyrate |
| glycerol | malonate | methanol | pyruvate | succinate |
| glycerol | malonate | methanol | pyruvate | proline |
| glycerol | malonate | methanol | pyruvate | hydroxy-isovalerate |
| glycerol | malonate | methanol | pyruvate | hydroxy-valerate |
| glycerol | malonate | methanol | pyruvate | 3-hydroxy-butyrate |
| malonate | methanol | pyruvate | succinate | proline |
| malonate | methanol | pyruvate | succinate | hydroxy-isovalerate |
| malonate | methanol | pyruvate | succinate | hydroxy-valerate |
| malonate | methanol | pyruvate | succinate | 3-hydroxy-butyrate |
| methanol | pyruvate | succinate | proline | hydroxy-isovalerate |
| methanol | pyruvate | succinate | proline | hydroxy-valerate |
| methanol | pyruvate | succinate | proline | 3-hydroxy-butyrate |
| pyruvate | succinate | proline | hydroxy- | hydroxy-valerate |
| | | | isovalerate | |
| pyruvate | succinate | proline | hydroxy- | 3-hydroxy-butyrate |
| | | | isovalerate | |
| succinate | proline | hydroxy- | hydroxy- | 3-hydroxy-butyrate |
| | | isovalerate | valerate | |

Other 6, 7, 8, 9, 10, 11, 12, 13, 14, and 15 metabolite combinations for Trisomy 18 can be readily determined by a skilled artisan using routine experimentation.

Collection of blood from a woman is performed in accordance with the standard protocol hospitals or clinics generally follow. An appropriate amount of peripheral blood, e.g., between 3-20 ml, is collected (and stored, if needed) according to standard procedure prior to further preparation. In addition to whole blood, the serum of a woman's blood is suitable for use in the methods of the present invention and can be obtained by well known methods. For example, serum is obtained through centrifugation following blood clotting. Centrifugation is typically conducted at an appropriate speed, e.g., 1,500-3,000×g, in a chilled environment, e.g., at a temperature of about 4-10° C.

A dried blood sample from a pregnant woman can be used to measure metabolites for assessing the risk of a fetus having Trisomy 21 or Trisomy 18. Blood is collected from a pregnant woman to be screened and transferred to filter paper where the blood dries, resulting in a spot, or spots, of dried blood on the filter paper. This method can also be used with other bodily fluids, including urine. For example, drops of urine, or other bodily fluid, from a pregnant woman can be placed on a specimen card and dried. The dried spots can then be analyzed by conventional immunological techniques, known to those of ordinary skill in the art, in a manner similar to the manner described herein with reference to the analysis of dried blood spots.

Analyzing dried blood samples or dried samples of other bodily fluids provides advantages in the transport and storage of such samples. By way of example, the dried blood spot samples on filter paper take up much less space than liquid blood samples in test tubes. Thus, less space is needed to store the samples, and the samples can be shipped by conventional mail or delivery services in small packages. A further advantage of using dried blood spots is that in the methods of the present invention is that a smaller volume of blood is collected from the pregnant woman than in the case where a liquid blood sample is to be analyzed. Since less blood is needed, it is possible to make the collection technique less invasive, and potentially less painful for the pregnant woman. Other advantages in shipping dried blood or urine spots on filter paper in comparison with liquid blood samples in tubes or vials are readily apparent to those of skill in the art.

The filter paper for preparing dried samples, which is also referred to as a specimen collection card, is commercially available from a variety of sources, including Whatman, Inc., and Schleicher & Schuell. Generally a 3 inch by 4 inch, or a 5 inch by 7 inch card is utilized to collect the samples, however the filter paper may be any size that is convenient for transporting, storing and/or indexing the dried bodily fluid samples. The filter paper can be of sufficient size to enable a technician or nurse to write the pregnant woman's name or other identifier as well as other information such as the date the sample was collected on the paper. For example, the filter paper (specimen collection card) can be a Schleicher & Schuell #903® 3 inch by 4 inch card pre-printed with circles to provide locations for the blood spots (application sites) and spaces to enter the patient's identification number, birth date, the date of collection of the sample and the physician's name. The filter paper can be provided with instructions and a lancet for a pregnant woman to collect her own blood.

The amount of blood taken from the pregnant woman should be sufficient to produce at least one spot on the filter paper approximately 10 millimeters in diameter. It is generally advantageous to produce more than one dried blood spot. Preferably, the amount of blood taken from the pregnant woman is sufficient to produce five to eight spots approximately 10 mm in diameter on the filter paper. It will be understood by those of ordinary skill in the art that the number of blood spots produced on a single piece of filter paper depends on the dimensions of the filter paper and the requirements of the physician and the clinical laboratory that will be analyzing the blood.

A variety of techniques for “spotting” blood on filter paper are known to the art. The choice of the particular technique utilized to produce the blood spots is a matter of choice to the person collecting the sample. Generally, a convenient site on the pregnant woman, preferably a finger tip, toe or ear lobe, is sterilized and then pricked with a sterile lancet. Lancets are commercially available from a variety of sources. An especially useful lancet is the Tenderlett® lancet manufactured and sold by Technidyne Corporation.

The drops of blood that form at the pricked site may be allowed to drip onto the filter paper to form the blood spots. Alternatively the pricked site may be placed in contact with the filter paper to produce the blood spots. The blood should dry on the filter paper prior to transport and/or storage.

The filter paper containing the blood spot is then analyzed to determine the pregnant woman's level of one or more metabolites utilized in the screening protocol. The filter paper containing the blood spot may be stored and/or transported prior to analysis.

In addition to blood, other bodily fluids obtained from a pregnant woman being assessed for a risk of carrying a fetus with Down syndrome or Trisomy 18 can be used to measure concentrations of metabolites in such fluids. In one embodiment, the bodily fluid is urine, such as the first morning urine, which a pregnant woman can collect herself. The urine can be collected in cups specially provided for urine collection or can also be spotted on a filter paper, as discussed above.

In cases when urine is used as a bodily fluid, an easy-to-use nomogram can be used to translate a spot urine metabolite concentration into an estimated 24-hour excretion of one or more metabolites being tested for Down syndrome or Trisomy 18.

The development of a set of easy-to-use nomograms provides a simple method for adjustment of a metabolite/creatinine ratio, which enables estimation of the 24-hour excretion of any metabolite with much greater accuracy than is possible without the adjustment. For example, the nomograms are used to adjust the ratio of the metabolite (e.g., glycerol) and creatinine concentrations to the estimated 24-hour creatinine excretion based upon one or more characteristics of the individual, such as age, gender, race, weight, lean body mass, muscle mass, adiposity, physical activity, or any combination thereof, to better estimate the 24-hour metabolite excretion.

A spot urine sample is obtained from a pregnant woman being assessed for a risk of carrying a fetus with Down syndrome or Trisomy 18 by any standard methods known in the art. The concentrations of a metabolite of interest and creatinine in the spot urine sample are determined by any of the methods described herein, such as mass spectroscopy or NMR. In cases of well established metabolites, the measurement of the metabolite concentration can be obtained from a standard clinical laboratory or a dipstick, if such is available. Then, a standard value for estimated 24-hour urine creatinine excretion is selected from an array of such standard values for 24-hour urine creatinine excretion. The values in the array are obtained from a nomogram that is a product of an equation that estimates 24-hour creatinine excretion from variables including subject's age, gender, race, weight, muscle mass, lean body mass, muscle mass, adiposity, physical activity, or a combination thereof. A standard value for estimated 24-hour excretion of the metabolite is then selected from an array of such standard values for 24-hour metabolite excretion. The values in the array are based upon the standard value for estimated 24-hour urine creatinine excretion determined in the previous step, the metabolite concentration and the creatinine concentration.

The mass of creatinine excreted by a pregnant woman being tested, who is not afflicted with any substantial challenge to homeostasis can be expected to remain reasonably constant over time. In other words, for any individual with stable renal function, the 24-hour urinary creatinine excretion is constant from day to day. This applies as well to individuals with impaired renal function, provided it is stable.

Determination of the amount of a metabolite that is excreted in the urine, or of changes in the amount excreted, is generally obtained by measuring the concentration of the metabolite in a 24-hour urine collection. For many metabolites, more convenient estimation of their excretion can be obtained by estimation of their excretion from a spot urine sample by assessing their concentration in the urine relative to the concentration of creatinine in that sample.

The 24-hour creatinine excretion, although constant from day to day in any given individual, differs considerably between individuals. Thus, for example, a 100 pound woman might have a urine creatinine excretion of 900 mg/day, whereas a 250 pound male might have a urine creatinine excretion of 2500 mg. Because of this considerable between-person variance in 24 hour urine creatinine excretion, to accurately estimate the 24-hour excretion of a metabolite from a metabolite/creatinine ratio, the ratio must be adjusted to take into account the amount of creatinine typically excreted by that individual in 24-hours. The usual method of determining 24-hour creatinine excretion, again, is a 24-hour urine collection, which is plagued by the impracticality of collecting urine for 24 hours and by inaccuracy in many cases due to under-collection. However, an estimate of the individual's 24-hour creatinine excretion, without any urine collection, is possible if one or more variables associated with between-subject variation in creatinine excretion are accounted for in determining the estimate of creatinine excretion. Such variables include lean muscle mass, which can be largely determined from gender, race, age, weight, muscularity or a combination thereof. The estimated 24-hour creatinine thus takes into account between-person differences, requires no urine collection, and, unlike actual 24-hour urine collections, its accuracy does not suffer from incomplete collections. The estimated 24-hour creatinine excretion determined in this fashion is used to adjust the measured metabolite/creatinine ratio to accurately predict 24-hour excretion of that metabolite.

This method provides an estimate for 24-hour urine creatinine and simple instructions for use to enable appropriate adjustment of the metabolite/creatinine ratio. It avoids the need for any blood sample, or for measurement of urine volume, while adding precision to raw metabolite/creatinine ratios. The adjusted estimate of 24-hour creatinine excretion is available in the form of databases, look-up tables or nomograms, for example. In preferred embodiments, these values are arrayed such that a “standard” value for a given subject, depending upon the subject's age, race, gender, weight, muscle mass, lean body mass and/or level of physical activity, can be selected from the array. Without wishing to be bound by a particular theory, this method of estimating 24-hour urine creatinine is thought to improve the accuracy of prediction of metabolite excretion because it bears a strong relationship to an individual's muscle mass, which is the source of creatinine. Since weight, gender and ethnicity are prominent determinants of total muscle mass, and are readily available measures, formulae can be created for estimating a subject's 24-hour creatinine excretion. An exemplary formula, is:

{y=1150 mg−407.4 mg (if female)+(5.7)(weight in pounds)−88 mg (if white)}

wherein y is a subject's estimated 24-hr creatinine excretion in mg. It will be appreciated that the artisan can readily refine the model by adding variables (e.g., gender, age, ethnicity such as Asian, Caucasian or African, lean muscle mass, adiposity, or level of physical activity), accumulating data on each variable, and deriving therefrom, by well-known methods of regression analysis, more powerful regression formulae.

Any equation so determined can be applied to estimate the 24-hour creatinine excretion of any individual, without limitation, by hand, or by means of a computer program, a look-up table or a nomogram. A nomogram is preferred since it can be used without the need for calculations by the subject.

The adjusted estimate of a pregnant woman's 24-hour excretion of one or more metabolites is thus readily calculated in two steps: first, by consulting the table of estimated 24-hour creatinine excretion values for pregnant women, and selecting a value corresponding to the individual's weight and age, and second, by using the selected value along with the values found for urinary metabolite concentration and urinary creatinine concentration in a spot urine sample, solving the following equation:

Subject's Metabolite Excretion=((Creatinine Excretion per Table)(Metabolite Conc.))/((Creatinine Conc.)×10).

Again, the equation can be solved, without limitation, by hand, or by means of a computer program, a look-up table or a nomogram.

The concentrations (or levels) of metabolites obtained from a bodily fluid of a pregnant woman being assessed for a risk of carrying a fetus with Trisomy 21 or Trisomy 18 can be measured using a variety of techniques well known in the art. Such methods include, but are not limited to, mass spectrometry (MS), nuclear magnetic resonance (NMR), immunoblot analysis, immunohistochemical methods (e.g., in situ methods based on antibody detection of metabolites), and immunoassays (e.g., ELISA). It should be noted that a method used to measure metabolites in a pregnant woman being assessed for a risk of carrying a fetus with Trisomy 21 or Trisomy 18 is the same method that is used to measure concentrations of corresponding metabolites in control subjects (i.e., pregnant women carrying fetuses with either Trisomy 21 or Trisomy 18, depending on the condition being tested and pregnant women carrying chromosomally normal fetuses).

Mass spectrometry (e.g., electrospray ionization or ESI mass spectrometry) can be used, for example, to determine the concentrations of metabolites in a maternal sample. In general, mass spectrometry involves ionizing a sample containing one or more molecules of interest, and then m/z separating and detecting the resultant ions (or product ions derived therefrom) in a mass analyzer, such as, without limitation, a quadrupole mass filter, quadrupole ion trap, time-of-flight analyzer, FT/ICR analyzer or Orbitrap, to generate a mass spectrum representing the abundances of detected ions at different values of m/z. See, e.g., U.S. Pat. Nos. 6,204,500, 6,107,623, 6,268,144, and 6,124,137, and articles, such as Wright et al., Prostate Cancer and Prostatic Diseases 2: 264-76 (1999); and Merchant and Weinberger, Electrophoresis 21: 1164-67 (2000), each of which is hereby incorporated by reference in its entirety.

Tandem mass spectrometry (e.g., using a quadrapole mass spectrometer) can be employed in the methods of the invention. As used herein “tandem mass spectrometry,” or “MS/MS” refers to a technique wherein a precursor ion or group of ions generated from a molecule (or molecules) of interest may be isolated or selected in an MS instrument, and these precursor ions subsequently fragmented to yield one or more fragment ions that are then analyzed in a second MS procedure. By careful selection of precursor ions, ions produced by certain metabolites of interest are selectively passed to the fragmentation chamber, where collision with atoms or molecules of an inert gas occurs to produce the fragment ions. Since both the precursor and fragment ions are produced in a reproducible fashion under a given set of ionization/fragmentation conditions, the MS/MS technique can provide an extremely powerful analytical tool. For example, the combination of filtration/fragmentation can be used to eliminate interfering substances, and can be particularly useful in complex samples, such as biological samples.

Ions can be produced using a variety of methods including, but not limited to, electrospray ionization (“ESI”), and matrix-assisted laser desorption ionization (“MALDI”).

Electrospray ionization, or ESI, mass spectrometry can be used to determine the expression level of one or more metabolites in a maternal sample. The term “electrospray ionization,” or “ESI,” as used herein refers to methods in which a solution is passed along a short length of capillary tube, to the end of which is applied a high positive or negative electric potential. Solution reaching the end of the tube is vaporized (nebulized) into a jet or spray of very small droplets of solution in solvent vapor. This mist of droplets flows through an evaporation chamber which may be heated to prevent condensation and to evaporate solvent. As the droplets get smaller the electrical surface charge density increases until such time that the natural repulsion between like charges causes ions as well as neutral molecules to be released.

For MALDI, the sample is mixed with an energy-absorbing matrix, which facilitates desorption of analyte (metabolite) molecules.

In methods such as MS/MS, where precursor ions are isolated for further fragmentation, collision-induced dissociation (“CID”) is often used to generate the fragment ions for further detection. In CID, precursor ions undergo fragmentation induced by energetic collisions with neutral molecules or atoms. Sufficient energy must be deposited in the precursor ion so that certain bonds within the ion can be broken due to increased vibrational energy.

NMR spectroscopy can also be used to determine concentrations of metabolites in a pregnant woman's sample who is being tested for probability of carrying a fetus with Down syndrome or Trisomy 18, and in samples which are used as controls. NMR is based on the magnetic properties of the nucleus of the constituent atoms that make up the metabolite. Exposure to radiofrequency (RF) energy will result in a change of energy state or orientation of these ‘nuclear magnets’. The exact frequency of RF energy needed to achieve this change in energy state is specific for a particular atomic element (Bothwell J H, Griffin J L. Biol Rev Camb Philos Soc. 2010 Oct. 24. doi: 10.1111/j.1469-185X.2010.00157.x.). When the RF energy pulse is turned off the nuclei returns to their resting position, thereby remitting the stored energy in the form of RF waves. The parameters of the RF waves emitted from the nuclei provides information on the chemical substances that are present in the sample being tested. The emitted RF waves are read as a plot of intensity on the Y-axis and frequency on the X-axis. These spectra are compared to internal standard substances placed in the specimen and existing databases to determine the identity and concentrations of metabolites within the specimens being tested. In some embodiments, the metabolite concentrations are analyzed using ¹H NMR.

The biological samples can be subjected to one or more sample preparation steps prior to analysis by mass spectrometry. For example, a serum sample can be enriched for target metabolites of interest using techniques known in the art, such as by concentrating the samples.

In some embodiments, samples are subjected to a liquid chromatography (LC) purification step prior to mass spectrometry. Methods of coupling liquid chromatography techniques to MS analysis are well known and widely practiced in the art. Traditional LC analysis relies on the chemical interactions between sample components and column packings, where laminar flow of the sample through the column is the basis for separation of the analyte of interest from the test sample. The skilled artisan will understand that separation in such columns is a diffusional process. Numerous column packings are available for chromatographic separation of samples, and selection of an appropriate separation protocol is an empirical process that depends on the sample characteristics, the metabolite of interest, the interfering substances present and their characteristics, etc. Various packing chemistries can be used depending on the needs (e.g., structure, polarity, and solubility of compounds being purified). For example, the columns can be polar, ion exchange (both cation and anion), hydrophobic interaction, phenyl, C-2, C-8, C-18 columns, polar coating on porous polymer, or others that are commercially available. During chromatography, the separation of materials is effected by variables such as choice of eluant (also known as a “mobile phase”), choice of gradient elution and the gradient conditions, temperature, etc.

A metabolite may be purified by applying a sample to a column under conditions where the metabolite of interest is reversibly retained by the column packing material, while one or more other materials are not retained. A first mobile phase condition can be employed where the metabolite of interest is retained by the column, and a second mobile phase condition can subsequently be employed to remove retained material from the column, once the non-retained materials are washed through. Alternatively, a metabolite can be purified by applying a sample to a column under mobile phase conditions where the metabolite of interest elutes at a differential rate in comparison to one or more other materials. As discussed above, such procedures can enrich the amount of one or more metabolites of interest relative to one or more other components of the sample.

Once the mass spectrometric or NMR analysis of the prepared sample has been completed, the quantities of the metabolites in the sample can be determined by integration of the relevant mass spectral peak areas, as known in the prior art. When isotopically-labeled internal standards are used, as described above, the quantities of the metabolites of interest are established via an empirically-derived or predicted relationship between metabolite quantity (which may be expressed as concentration) and the area ratio of the metabolite and internal standard peaks at specified transitions. Other implementations of the assay can utilize external standards or other expedients for metabolite quantification.

It is obvious to those skilled in the art that a cut-off can be established to determine whether a patient is at increased risk of carrying a fetus with Trisomy 18 and/or Trisomy 21. This cut-off may be established by the laboratory, the physician or on a case by case basis by each patient. The cut-off level can be based on several criteria including the number of women who would go on for further invasive diagnostic testing, the average risk of carrying a Down syndrome fetus of all the women who go on for further invasive diagnostic testing, a decision that any woman whose patient specific risk is greater than a certain risk level such as 1 in 400 should go on for further invasive diagnostic testing or other criteria known to those skilled in the art. The cut-off level could be established using a number of methods, including: percentiles, mean plus or minus standard deviation(s); multiples of median value; patient specific risk or other methods known to those skilled in the art.

For the purposes of the discriminant analysis, an assumption is made as to the prior probability of Down syndrome in the general unselected population. Generally, the prior probability is approximately 1 in 800. For the multivariate discriminant analysis a decision is made as to what risk cutoff level constitutes a positive test result. For example, if it is desirable to perform further diagnostic tests on a pregnant woman who has a 1 in 400 or greater possibility of carrying a Down syndrome fetus, then when the results of the discriminant analysis indicate that a pregnant woman has a 1 in 400 or greater possibility of carrying a Down syndrome fetus, the pregnant woman is considered to have a positive test result. If a positive test result is indicated, the patient should be counseled about further diagnostic tests to confirm the presence of Down syndrome.

As obvious to one skilled in the art, in any of the embodiments discussed above, changing the risk cut-off level of a positive or using different a priori risks which may apply to different subgroups in the population could change the results of the discriminant analysis for each patient. Accordingly, if by the methods of the present invention a pregnant woman being assessed for a risk of carrying a fetus with Trisomy 18 or Trisomy 21 has a greater probability than the cut-off value, she may be advised to undergo further testing, such as amniocentesis or CVS.

The methods for predicting a pregnant woman's risk for carrying a fetus with Down syndrome or Trisomy 18 can include factors other than metabolite concentrations in determining such risk. These factors include, without limitation, maternal age and/or measurements of nuchal translucency for both Trisomy 21 and Trisomy 18.

Accordingly, the present invention is also directed to a method for determining a pregnant woman's risk of carrying a fetus with Down syndrome, wherein the method comprises measuring concentrations of one or more metabolites selected from the group consisting of 2-hydroxy-butyrate, 3-hydroxy-isovalerate, acetamide, arginine, choline, glycerol, glycine, propylene glycol, carnitine, creatinine, phenylalanine and leucine in the pregnant woman's bodily fluid. The pregnant woman's one or more metabolite concentrations are compared to the corresponding one or more metabolite concentrations obtained from pregnant women carrying Down syndrome fetuses and the corresponding one or more metabolite concentrations obtained from pregnant women carrying chromosomally normal fetuses. All metabolite concentrations are measured at the same or similar gestational age. The pregnant woman's risk of carrying a fetus with Down syndrome is predicted based on age of the pregnant woman and change in concentration of one or more metabolites between the pregnant woman and the corresponding one or more metabolites from the pregnant women carrying chromosomally normal fetuses, wherein increased age of the pregnant woman and a statistically significant change in concentration of one or more metabolites between the pregnant woman and the corresponding one or more metabolites from the pregnant women carrying chromosomally normal fetuses indicates a greater probability of carrying a fetus with Down syndrome. For example, increased maternal age and a statistically significant change in concentration of one or more metabolites selected from the group consisting of carnitine, acetamide and 2-hydroxy-butyrate between the pregnant woman and the corresponding one or more metabolites from the pregnant women carrying chromosomally normal fetuses can be used to predict a risk of Trisomy 21 in a fetus.

“Nuchal translucency” or NT is the amount of fluid under the skin at the back of the fetal neck as measured by first-trimester ultrasound, and is a strong predictor of the risk of chromosomal abnormality. An increase in the amount of fluid collection correlates with an increased risk of Down syndrome and Trisomy 18. ΔNT (“delta nuchal translucency”) or the difference between the measured amount of fluid and that expected in normal fetuses of the same gestational age is used as a measure of whether the amount of nuchal fluid is increased (positive ΔNT value) or decreased (negative ΔNT value) compared to what would be expected for normal fetuses at this gestational age (gestational age represented by CRL measurement). A positive value for ΔNT thereby indicates an increased risk of fetal chromosomal abnormality while a negative value correlates with a decreased risk.

A method for determining a pregnant woman's risk of carrying a fetus with Down syndrome comprises measuring concentrations of one or more metabolites selected from the group consisting of 2-hydroxy-butyrate, 3-hydroxy-isovalerate, acetamide, arginine, choline, glycerol, glycine, propylene glycol, carnitine, creatinine, phenylalanine and leucine in the pregnant woman's blood or urine. The pregnant woman's one or more metabolite concentrations are compared to the corresponding one or more metabolite concentrations obtained from pregnant women carrying Down syndrome fetuses and pregnant women carrying chromosomally normal fetuses. All metabolite concentrations are measured at the same or similar gestational age. Nuchal translucency of the pregnant woman's fetus is measured and compared to nuchal translucency of the chromosomally normal fetuses at the same or similar gestational age. The pregnant woman's risk of carrying a fetus with Down syndrome is predicted, wherein the statistically significant change in concentration of the one or more metabolites between the pregnant woman and the corresponding one or more metabolites from the pregnant women carrying chromosomally normal fetuses and increased nuchal translucency of the pregnant woman's fetus as compared to the nuchal translucency of the chromosomally normal fetuses indicate greater probability of carrying a fetus with Down syndrome. For example, increased nuchal translucency and a statistically significant change in concentration of creatinine between the pregnant woman and the corresponding creatinine concentration from the pregnant women carrying chromosomally normal fetuses can be used to predict a risk of a fetus having Trisomy 21.

A method for determining a pregnant woman's risk of carrying a fetus with Trisomy 18 comprises measuring concentrations of one or more metabolites selected from the group consisting of 2-hydroxy-butyrate, acetate, choline, citrate, creatinine, ethanol, formate, glycerol, malonate, methanol, pyruvate, succinate, proline, hydroxy-isovalerate, hydroxy-valerate, and 3-hydroxy-butyrate in the pregnant woman's bodily fluid. The pregnant woman's one or more metabolite concentrations are compared to the corresponding one or more metabolite concentrations obtained from pregnant women carrying fetuses with Trisomy 18 and to the corresponding one or more metabolite concentrations obtained from pregnant women carrying chromosomally normal fetuses. All metabolite concentrations are measured at the same or similar gestational age. The pregnant woman's risk of carrying a fetus with Trisomy 18 is predicted based on age of the pregnant woman and change in concentration of one or more metabolites between the pregnant woman and the corresponding one or more metabolites from the pregnant women carrying chromosomally normal fetuses, wherein increased age of the pregnant woman and statistically significant change in concentration of one or more metabolites indicates a greater probability of carrying a fetus with Trisomy 18. For example, increased maternal age and a statistically significant change in concentration of one or more metabolites selected from the group consisting of phenyalanine, choline, carnitine, and acetamide between the pregnant woman and the corresponding one or more metabolites from the pregnant women carrying chromosomally normal fetuses can be used to predict the risk of Trisomy 18 in a fetus.

A method for determining a pregnant woman's risk of carrying a fetus with Trisomy 18 comprises measuring concentrations of one or more metabolites selected from the group consisting of 2-hydroxy-butyrate, acetate, choline, citrate, creatinine, ethanol, formate, glycerol, malonate, methanol, pyruvate, succinate, proline, hydroxy-isovalerate, hydroxy-valerate, and 3-hydroxy-butyrate in the pregnant woman's bodily fluid. The pregnant woman's one or more metabolite concentrations are compared to the corresponding one or more metabolite concentrations obtained from pregnant women carrying fetuses with Trisomy 18 and to the corresponding one or more metabolite concentrations obtained from pregnant women carrying chromosomally normal fetuses. All metabolite concentrations are measured at the same or similar gestational age. Nuchal translucency of the pregnant woman's fetus is measured and compared to nuchal translucency of the chromosomally normal fetuses at the same or similar gestational age. The pregnant woman's risk of carrying a fetus with Trisomy 18 is predicted, wherein the statistically significant change in concentration of one or more metabolites between the pregnant woman and the corresponding one or more metabolites from the pregnant women carrying chromosomally normal fetuses and increased nuchal translucency of the pregnant woman's fetus as compared to the nuchal translucency of the chromosomally normal fetuses indicate greater probability of carrying a fetus with Trisomy 18. For example, increased nuchal translucency and a statistically significant change in concentration of choline between the pregnant woman and the corresponding choline concentration from the pregnant women carrying chromosomally normal fetuses can be used for predicting Trisomy 18 in a fetus.

Nuchal translucency (ultrasound measurement of the amount of fluid at the back of the fetal neck) is measured using standard methods in the art. Nuchal translucency is measured between 11⁺⁰ and 13⁺⁶ weeks of pregnancy; however, nuchal translucency can be measured either before or after obtaining a pregnant woman's bodily fluid sample for measuring metabolite concentrations.

The particulars for performing the analyses involved in evaluating a pregnant woman's risk for carrying a fetus with Down syndrome or Trisomy 18 are described in the Examples. By way of example and not of limitation, the calculation of probability of the fetus having Down syndrome or Trisomy 18 can be derived using logistic regression analysis. In this mathematical equation, a number of potential predictor variables which can either be numerical (age in years, body weight, body mass index (BMI)) or categorical (e.g. race/ethnicity, gender, the presence of other disorders such as diabetes) are analyzed to find the optimal combination of variables that will most accurately predict an outcome of interest, e.g. chromosomal abnormality. The results of the logistic regression analysis can be converted to a format that expresses the probability of the particular outcome. Such formulas for Down syndrome and Trisomy 18 are shown in the Examples.

Control samples are appropriately matched to the pregnant women being assessed for a risk of carrying a fetus with Trisomy 18 or Trisomy 21 as is standard in the art. As such, the bodily fluids obtained from pregnant women being tested and controls (pregnant women carrying fetuses with either Trisomy 18 or Trisomy 21 and carrying chromosomally normal fetuses) are preferably the same (e.g., blood vs. blood), the gestational age of pregnant women being tested and controls is the same or similar (i.e., in the same range (e.g., 10-13 weeks of gestation)), concentrations of metabolites are measured using the same techniques, etc.

The screening methods of the invention can also be combined with existing screening techniques for the detection of Trisomy 21. Thus, the diagnostic methods described herein can be combined with an examination of one or more known biomarkers for Trisomy 21, such as, for example, one or more of serum biomarkers PAPP-A, alpha-fetoprotein (AFP), human chorionic gonadotropin (beta-hCG), unconjugated estriol (uE3), and inhibin A, or biomarkers for Trisomy 18, such as, e.g., AFP, estriol and hCG.

“Normalized” refers to data mathematically adjusted by a factor such that the elements of the factored dataset are more readily compared than the elements of the unfactored dataset. For example, each normalized metabolite concentration value can be an estimated average (mean or median) of observed metabolite concentrations from a population of pregnant women carrying chromosomally normal fetuses and being of similar age, race, weight, BMI, etc.

Another aspect of the invention is an article of manufacture such as a computer readable medium encoded with machine-readable data and/or a set of instructions, where the instructions can be carried out by a computer or a processing system. Such a computer readable medium can be a conventional compact disk read only memory (CD-ROM) or a rewritable medium such as a magneto-optical disk which is optically readable and magneto-optically writable. The computer readable medium can be prepared by available procedures. For example, the computable readable medium can have a suitable conventional substrate and a suitable conventional coating, usually on one side of the substrate.

In the case of CD-ROM, as is well known, a reflective coating can be employed that is impressed with a plurality of pits to encode the machine-readable data. The arrangement of pits is read by reflecting laser light off the surface of coating. A protective coating, which preferably is substantially transparent, is used on top of coating that has a plurality of pits.

In the case of a magneto-optical disk, as is well known, the coating has no pits, but has a plurality of magnetic domains whose polarity or orientation can be changed magnetically when heated above a certain temperature, as by a laser. The orientation of the domains can be read by measuring the polarization of laser light reflected from coating. The arrangement of the domains encodes data, for example, normalized metabolite concentration values measured in a bodily fluid of pregnant women, such as blood or urine, as described above.

Data capable of facilitating determination of a pregnant woman's risk for carrying a fetus with Down syndrome or Trisomy 18 is stored in a machine-readable storage medium. Executable code can also be included in the machine-readable medium that is capable of predicting the pregnant woman's risk for carrying a fetus with Down syndrome or Trisomy 18 when the medium is used in conjunction with a computer or processor. For example, the machine readable medium, used in conjunction with a computer or processor can determine a pregnant woman's likelihood for carrying a fetus with Down syndrome or Trisomy 18 after an individual enters data relating to the pregnant woman's bodily fluid concentrations of one or more metabolites.

The term “article of manufacture” as used herein refers to a kit or a computer readable medium (e.g., computer chip or magnetic storage medium such as hard disk drives, floppy disks, tape), optical storage medium (e.g., OD-ROMs, optical disks, etc.), volatile and non-volatile memory devices (e.g., EEPROMs, ROMs, PROMs, RAMs, DRAMs, SPAMs, firmware. programmable logic, etc.). Code and data in the computer readable medium is accessed and executed by a processor. The code and/or data in which implementations are made may further be accessible through a transmission media or from a file server over a network. Of course, those skilled in the art will recognize that many modifications may be made to this configuration without departing from the scope of the implementations and that the article of manufacture may comprise any information bearing medium known in the art.

The article of manufacture and the computer-readable medium are non-transitory, such that they comprise all such articles of manufacture and computer-readable media except for a transitory, propagating signal.

Having described the invention in detail, it will be apparent that modifications and variations are possible without departing from the scope of the invention defined in the appended claims.

### EXAMPLES

The following non-limiting examples are provided to further illustrate the present invention.

### NMR Sample Preparation

Plasma samples contain a substantial portion of large molecular weight proteins and lipoproteins, which can affect the identification and quantification of small molecule metabolites by NMR spectroscopy. To prevent this, a step to remove plasma proteins (deproteinization) was added to the protocol. There are several routes to plasma deproteinization, including organic solvent (acetonitrile, methanol, isopropanol) precipitation, as well as diffusion editing. An ultrafiltration protocol similar to that described by Tiziani et al. (Tiziani S et al., Anal Biochem. 2008; 377:16-23; Weljie A M et al., Anal Chem. 2006; 78:4430-42) yielded excellent spectra resulting in metabolite concentrations that closely matched known values measured using standard clinical chemistry techniques.

Prior to filtration, 3 KDa cut-off centrifugal filter units (Amicon Microcon YM-3) were rinsed three times each with 0.5 mL of water and centrifuged at 10,000 rpm for 30 minutes to remove residual glycerol bound to the filter membranes. 350 μL aliquots of each plasma sample were then transferred into the centrifuge filter devices. The samples were then spun at a rate of 10,000 rpm for 20 minutes to remove macromolecules (primarily proteins and lipoproteins) from the sample. The subsequent filtrates were then checked visually for an indication that the membrane was compromised. For those samples where the membrane was compromised, the filtration process was repeated with a different filter and the filtrate was inspected again. The subsequent filtrates were collected and the volumes were recorded. If the total volume of the sample was under 300 μL, an appropriate amount from a 50 mM monobasic sodium phosphate buffer (pH 7) was added to the sample until the total volume was 300 μL. Subsequently, 35 μL of deuterium oxide and 15 μL of a standard buffer solution (11.667 mM DSS [disodium-2,2-dimethyl-2-silapentane-5-sulphonate], 730 mM imidazole, and 0.47% NaN₃ in water) were added to the sample. The plasma samples (350 μL) were then transferred to standard Shigemi microcell NMR tubes for subsequent spectral analysis.

### NMR Spectroscopy

All ¹H-NMR spectra were collected on a 500 MHz Inova (Varian Inc., Palo Alto, Calif.) spectrometer equipped with either a 5 mm HCN Z-gradient or PFG Varian cold-probe. ¹H-NMR spectra were acquired at 25° C. using the first transient of the NOESY-presaturation pulse sequence, which was chosen for its high degree of quantitative accuracy. Spectra were collected with 256 transients and 8 steady state scans using a 4 second acquisition time and a 1.5 second recycle delay.

### NMR Compound Identification and Quantification

All free induction decays (FIDS) were zero-filled to 64k data points and subjected to line broadening of 0.5 Hz. The singlet produced by the DSS methyl groups was used as an internal standard for chemical shift referencing (set to 0 ppm) and for quantification. All ¹H-NMR spectra were processed and analyzed using the Chenomx NMR Suite Professional software package version 7.0 (Chenomx Inc., Edmonton, AB). The Chenomx NMR Suite software allows for qualitative and quantitative analysis of an NMR spectrum by manually fitting spectral signatures from an internal database to the spectrum. Specifically, the spectral fitting for each metabolite was done using the standard Chenomx 500 MHz metabolite library. Typically 90% of all visible peaks were assigned to a compound and more than 90% of the spectral area could be routinely fitted using the Chenomx spectral analysis software. Most of the visible peaks were annotated with a compound name. It has been previously shown that this fitting procedure provides absolute concentration accuracies of 90% or better. Each spectrum was processed and analyzed by at least two NMR spectroscopists to minimize compound misidentification and misquantification. Sample spiking was used to confirm the identities of assigned compound, which involves the addition of 20-200 μM of the suspected compound to selected clostridium samples and examination of whether the relative NMR signal intensity changed as expected.

### Statistical Analysis of Metabolic Data and Data Normalization.

The data were adjusted to achieve a normal or Gaussian distribution of metabolite concentrations. This permits the use of certain statistical tests that require normal distribution. A technique called Pareto scaling was used to achieve normalization. Metabolite concentrations were mean-centered, i.e. expressed with reference to the overall mean value and then divided by the standard deviation of each of the metabolites.

Principle components analysis (PCA) was used as a statistical technique for reducing (“dimensional reduction”) the number of metabolites (from a large original group of metabolites) that significantly account for the variance (difference) between the diseased and normal groups to a small number. The predictors are displayed on a 2- or 3-dimensional rather than on a high dimensional (>3) chart that would be required for the large number of metabolites available. Each principal component (metabolite set) displayed accounts for a significant percentage of the variance between the groups being studied e.g. Trisomy 18 vs. normal group or Trisomy 21 vs. normal group. Often a small number of principal components can account for a high percentage (e.g. >90%) of the variance between the two groups. In such circumstances, plotting the information on a three dimensional graph represents an easy means of visualizing the separation between the groups (Sumner L W et al., Methods Mol Biol. 2007; 406:409-36).

Partial least squares—discriminant analysis (PLS_DA) is a further method that was used to enhance the separation between the groups. The PCA components are rotated such that maximum separation between the groups is obtained (Wishart D S., Methods Mol Biol. 2010; 593:283-313).

Permutation analysis was used to confirm that the separation achieved between groups was not due to chance but was statistically significant. Random relabeling of the metabolomic data is performed thousands of times and PLS_DA was systematically repeated.

Table 1 shows eight metabolites for which the concentrations in maternal first trimester plasma was significantly different for the Down syndrome vs. normal control pregnancies. The concentrations of 3 metabolites were increased and were decreased for 5 metabolites in the Down syndrome group. These metabolite concentrations were determined from maternal blood samples obtained from pregnant women between 11⁺⁰ to 13⁺⁶ weeks of gestation at the time of sample collection, wherein the women later gave birth to a fetus with Down syndrome.

TABLE 1
Metabolite Concentrations for Down Syndrome vs. Unaffected Controls

| | Concentrations | Concentrations | |
|---|---|---|---|
| | Down syndrome | Control | |
| Metabolite | Mean (SD) | Mean (SD) | p-value |
| 2-OH-butyrate | 29.8 (12.8) | 22.0 (10.1) | <0.02 |
| 3-OH-isovalerate | 10.4 (2.8) | 8.8 (2.0) | 0.02 |
| Acetamide | 19.2 (15.0) | 10.8 (7.0) | 0.05 |
| Arginine | 108.3 (16.0) | 126.8 (31.2) | <0.005 |
| Choline | 9.9 (2.2) | 183.7 (322.8) | 0.001 |
| Glycerol | 210.2 (121.8) | 399.6 (450.8) | <0.02 |
| Glycine | 214.9 (38.5) | 260.5 (118.4) | <0.04 |
| Leucine | 72.1 (20.5) | 108.4 (86.3) | 0.01 |

Table 2 shows a total of twelve metabolites whose concentrations were significantly different in Trisomy 18 compared to normal groups. Of these metabolites, 2 were increased and 10 were decreased in concentrations in Trisomy 18 compared to normals. These metabolite concentrations were determined from maternal blood samples obtained from pregnant women between 11⁺⁰ to 13⁺⁶ weeks of gestation at the time of sample collection, wherein the women later gave birth to a fetus with Trisomy 18.

TABLE 2
Metabolite Concentrations in Trisomy 18 and Unaffected Controls

| | Concentrations | Concentrations | |
|---|---|---|---|
| | Trisomy 18 | Control | |
| Metabolite | Mean (SD) | Mean (SD) | p-value |
| 2-OH-butyrate | 29.8 (14.6) | 22.0 (10.1) | <0.02 |
| Acetate | 25.6 (21.2) | 53.7 (56.9) | 0.008 |
| Choline | 47.7 (146.7) | 183.7 (322.8) | 0.03 |
| Citrate | 66.9 (17.1) | 80.7 (21.7) | 0.026 |
| Creatinine | 36.1 (12.0) | 38.0 (14.3) | 0.04 |
| Ethanol | 31.0 (17.7) | 58.1 (30.6) | <0.001 |
| Formate | 14.6 (7.0) | 20.8 (16.8) | <0.05 |
| Glycerol | 139.6 (161.5) | 399.6 (450.8) | 0.002 |
| Malonate | 12.8 (6.8) | 19.3 (11.6) | 0.01 |
| Methanol | 184.8 (78.1) | 267.4 (136.2) | 0.006 |
| Pyruvate | 88.8 (29.4) | 71.4 (29.0) | <0.05 |
| Succinate | 6.6 (6.9) | 12.9 (15.4) | <0.04 |

### Calculation of Probability of Chromosomal Anomaly

The calculation of probability of the fetus having a disorder such as Down syndrome or Trisomy 18 can be derived using logistic regression analysis. In this mathematical equation, a number of potential predictor variables which can either be numerical (age in years) or categorical (e.g. race, gender) are analyzed to find the optimal combination of variables that will most accurately predict an outcome of interest, e.g. chromosomal abnormality. The results of the logistic regression analysis can be converted to a format that expresses the probability of the particular outcome, such as

P_{Down syndrome}=1/1+e^{-(β1×1+β2×2+β3×3 . . . βn×n)}

wherein x₁, x₂, x₃ etc. . . . represent the levels or concentrations of individual metabolites that were found to be significant predictors of the outcome of interest (Down syndrome in the fetus). β₁, β₂, β₃ are the so called R-coefficients. These are determined from the results of the logistic regression analysis, and represent the extent to which the probability of the outcome (e.g. Down syndrome in the fetus) changes for each unit change in that particular predictor variable. The same equation can be applied to calculating probability of Trisomy 18 in a fetus, namely

P_{Trisomy 18}=1/1+e^{-(β1×1+β2×2+β3×3 . . . βn×n)}

For example, if x₁ in the above probability equations was maternal age in years, then β₁ would represent the degree to which the probability of the fetus having Down syndrome “P_{Down syndrome}” changes with each one year change in maternal age. Thus, knowing the values for ‘x’ in a particular individual and the ‘R’ values from a logistic regression performed in a population of pregnancies consisting of fetuses both with and without Down syndrome, the probability of fetal Down syndrome in an individual pregnancy being tested can be derived. To determine the accuracy of a group of predictors for an outcome of interest, the individual probabilities of the outcome are calculated for cases in the groups with and without the outcome of interest. Different threshold probabilities e.g. 1/10, for the outcome can be used individually as screening tests to determine the percentage of affected cases with a calculated probability of Down syndrome 1/10 (sensitivity) and the percentage of normal cases in the population group with calculated probability values less than the threshold, i.e. < 1/10, defined as the specificity. A series of threshold DS probability values are used e.g. ≧ 1/10, ≧ 1/20, ≧ 1/30 etc, and the corresponding sensitivity and specificity for each determined, thereby generating a series of paired sensitivity and specificity values, with each pair corresponding to a particular threshold. A receiver operating characteristic (ROC) curve which is a plot of data points with sensitivity values on the Y-axis and false positivity rate (=1−specificity) on the X-axis is generated. The area under the ROC curves (AUC) indicates the accuracy of the test in identifying normal from abnormal cases (Hanley J A, McNeil B J. Radiology. 1982; 143(1):29-36; Peirce J C, Cornell R G. Med Decis Making. 1993; 13:141-51). The closer the area under the ROC curve is to 1, the greater the accuracy of the test. An area under ROC=1 signifies a perfect test in which all normal and affected individuals are correctly identified by the test.

Another commonly used method of risk calculation, namely multivariate Gaussian analysis can be used for risk calculation. Such a method is employed when the distribution or spread of measured concentration values of an individual biomarker in a group undergoing testing is symmetrical or bell-shaped. This implies an equal proportion of high and low measurement values are obtained, and this distribution is called a normal Gaussian distribution. The calculation of risk-probability based on Gaussian mathematics is well known and widely used in the art (Reynolds T M, Penney M D. Ann Clin Biochem. 1990; 27:452-8; Royston P, Thompson S G. Stat Med. 1992; 11:257-68). The multivariate Gaussian model uses mean (average) standard deviation and correlation coefficient between a normal population and one having the disorder. By knowing the distributions of biomarker values in a normal and an affected population, the risk or probability of an individual being affected can be determined by comparing the measured level of the biomarker in that individual to its known frequency in a normal and affected population groups.

### Specimen Collection

Maternal plasma was collected prospectively from pregnant women between 11⁺⁰ and 13⁺⁶ weeks of gestation at King's College Hospital, London, U.K. The diagnosis of Trisomy 18 and 21 was made based on genetic testing (karyotype) of fetal cells obtained prenatally (amniocentesis or CVS) or in the newborn period after birth. Table 3 shows the number of Trisomy 18, Trisomy 21 and normal cases that were evaluated.

TABLE 3
Crown Rump Length (Gestational Age) and Chromosomally Abnormal vs.
Normal Groups

| Chromosome | Number of | Aneuploidy Group | Normal Group | p- |
|---|---|---|---|---|
| Anomaly | Cases | CRL Mean (SD) | CRL Mean (SD) | value |
| Down | 16 | 59.7 (6.9) | 64.8 (8.8) | 0.4 |
| Syndrome | | | | |
| Trisomy 18 | 16 | 66.3 (7.6) | 64.8 (8.8) | 0.5 |
| Normals | 43 | — | — | — |
| (Unaffected) | | | | |

The mean gestational length represented by crown-to-rump length (CRL) was 45-84 mm, also shown in Table 3. This is equivalent to a gestational age of approximately 11⁺⁰ and 13⁺⁶ weeks at the time of specimen collection. There were no statistically significant differences in the CRL or gestational ages of the Trisomy 18 and 21 fetuses when compared to controls at the time of blood specimen collection.

Sample of blood were obtained from pregnant women, plasma extracted and then frozen and stored at−80° C. Briefly, blood samples were collected in anticoagulated tubes, then centrifuged at 3000 g (g-force) for 70 minutes to separate the cells from the liquid portion of the blood or plasma. The liquid portion of the blood was then aspirated, placed in a separate tube and kept in a −80° C. freezer for later laboratory analysis. The stored specimens were subsequently thawed and subjected to NMR spectroscopy.

NMR based metabolomic analysis was subsequently performed. Chemical substances (metabolites) were identified as separate resonance peaks on the NMR spectroscopic output. The intensity of the peaks correlated with the concentration of the substance or metabolite. Using databases of metabolomic standards, the identity of the metabolite represented by the peak was determined.

Metabolites whose concentrations were found to differ significantly in samples from women carrying Down syndrome fetuses and Trisomy 18 fetuses compared to normals (samples from pregnant women carrying chromosomally normal fetuses) were used to calculate the probability of fetal Down syndrome and Trisomy 18 in the two groups being studied.

### Individual Risk of Chromosome Anomaly

This calculation was based on logistic regression analysis leading to identification of the significant predictors among metabolites, ultrasound measurement of nuchal fluid collection (nuchal translucency), maternal demographic and other characteristics. The probability of an affected fetus in a given pregnancy is calculated from the probability equation:

P_{Down syndrome}=1/1+e^{-(β1×}₁^{+β2×}₂^{+β3×}₃ ^{. . . βn×}ₙ⁾

where ‘x’ refers to the magnitude or quantity of the particular predictor and “R” or R-coefficient refers to the magnitude of change in the probability of the outcome (Down syndrome) for each unit change in the level of the particular predictor, the R and ‘x’ values are derived form the results of the logistic regression analysis. The same equation applies for the calculation of Trisomy 18 probability in a fetus.

As noted above, risk probabilities can be calculated based on the logistic regression or multivariate Gaussian analysis method. The diagnostic accuracy of the method can be determined by calculating the area under the ROC curve, generated from the test data. Recently, newer and reportedly more accurate methods for determining the accuracy of screening tests such as Genetic computing have been reported (Kell D et al., Plant Physiol 0.2001; 126:943-51; Kell D., Bioinformatics World January-February 2002: 16-18). Genetic computing using the Gmax statistical program (The Gmax version 10.10.22: <http://www.thegmax.com/>) was used to independently evaluate the accuracy of the algorithms used herein.

### Example 1

### Maternal Age, Metabolite Concentrations and CRL for the Prediction of Fetal Chromosomal Abnormality

Logistic regression analysis, which included the metabolites listed in Table 1, maternal age and crown-rump length (CRL) was performed for the prediction of Down syndrome in the fetus. Some of the predictors were maternal age, 2-hydroxy-butyrate, acetamide and carnitine concentrations plus CRL measurement. The probability of Down syndrome in a fetus was thereby calculated using the derived regression equation:

P_{Down syndrome}=1/1+e^{-(0.274 MA+0.088 2-OH butyrate-0.241 carnitine+0.163 acetamide-0.195 CRL)}

where MA refers to maternal age in years, and the R-coefficients denoted therein were multiplied by the concentration of the corresponding metabolites.
These markers had 86.4% accuracy for correctly identifying Down syndrome and normal cases. The area under the ROC curve was highly significant, as shown in Table 4.

TABLE 4
Maternal Age and Metabolite Concentrations and Prediction of Fetal
Chromosomal Abnormality: Logistic Regression Method

| | Area Under the ROC | |
|---|---|---|
| Chromosome Anomaly | (95% CI) | p-value |
| Down Syndrome* | 0.94 (0.89, 1.00) | <0.001 |
| Trisomy 18** | 0.91 (0.84, 0.99) | <0.001 |

*maternal age, CRL, carnitine, acetamide, and 2-OH-butyrate
**maternal age, 2-OH butyrate, creatinine and ethanol

### Prediction of Trisomy 18 and Normal Cases

Individual probability of a fetus having Trisomy 18 was derived from the logistic regression analysis based equation.

P_{Trisomy 18}=1/1+e^{-(0.30 MA+0.084 2-OH butyrate-0.082 creatinine+0.047 ethanol)}

wherein MA is maternal age, and the R-coefficients denoted therein were multiplied by the concentration of the corresponding metabolites. When combined, 2-hydroxy-butyrate, creatinine and ethanol were found to have an 84.7% accuracy for correctly identifying normal and Trisomy 18 cases. The area under the ROC curve was highly significant as shown in Table 4.

### Genetic Computing

Using Genetic computing, a combination of propylene glycol, choline, carnitine, acetamide, phenylalanine and 2-hydroxy-butyrate plus maternal age predicted accurately Down syndrome risk. For Trisomy 18, the combination of glycol, proline, hydroxy-isovalerate, 3-hydroxy-butyrate and pyruvate accurately predicted Trisomy 18. With these particular metabolites, maternal age was not a significant contributor to Trisomy 18 prediction. The AUC of the ROC curves for these algorithms were highly significant and are shown in Table 5.

TABLE 5
Prediction of Fetal Chromosomal Abnormalities using Maternal Age and
Metabolite Concentrations: Genetic computing

| Chromosome Anomaly | Area Under the ROC Curve |
|---|---|
| Down Syndrome* | 0.96 |
| Trisomy 18** | 0.94 |

*Proplene glycol, choline, carnitine, acetamide, phenylanine, 2-OH-butyrate and maternal age
**Glycerol, proline, OH-isovalerate, 3-OH-butyrate, pyruvate (maternal age not a significant predictor)

In the case of Trisomy 21 prediction, phenyalanine contributed 50.55%, choline 18.8%, carnitine 10.43%, acetamide 10.58% to the prediction while maternal age contributed only 1.26% to the discrimination of the groups. Both traditional and newer statistical approaches confirmed the high diagnostic accuracy of the new metabolites.

### Example 2

### Prediction of Fetal Chromosomal Anomaly Based on Biochemical Analytes and Nuchal Translucency

To determine the probability of Down syndrome and Trisomy 18, variables tested in the logistic regression were maternal age, CRL, metabolite concentrations and ΔNT. In one example, significant predictors for Down syndrome based on the logistic regression analysis were creatinine concentrations and ΔNT. Individual probability for Down syndrome in the fetus based on these two variables were determined from the following derived equation:

P_{Trisomy 21}=1/1+e^{-(0.10 creatinine+4.181 ΔNT)}

In one example, significant predictors for Trisomy 18 were choline concentration and A NT. The logistic regression equation for the prediction of individual probability of Trisomy 18 based on these two variables was as follows:

P_{Trisomy 18}=1/1+e^{-(0.469 creatinine+322.272 ΔNT)}

Neither maternal age or gestational age (CRL) contributed significantly to the prediction of either chromosome abnormality in these particular variable/metabolite combinations. A highly accurate prediction of Down syndrome and Trisomy 18 was achieved with the above variables as shown in Table 6.

TABLE 6
Metabolites and Nuchal Translucency for the Prediction of Chromosomal
Anomaly in the Fetus: Logistic regression Method

| | Area Under the ROC | |
|---|---|---|
| Chromosome Anomaly | (95% CI) | p-value |
| Down Syndrome* | 0.96 (0.87, 1.00) | <0.001 |
| Trisomy 18** | 1.00 (1.00, 1.00) | <0.001 |

*creatinine
**choline

### Genetic Computing

A parsimonious model consisting of phenyalanine and choline concentrations and ΔNT had a high accuracy for the prediction of Down syndrome while a model consisting of glycerol and hydroxy-isovalerate concentrations combined with ΔNT was highly accurate for the prediction of Trisomy 18. Table 7 shows the AUC of the ROC curves for Down syndrome and Trisomy 18 prediction using nuchal translucency measurement and the above mentioned metabolite concentrations.

TABLE 7
Metabolites and Nuchal Translucency for the Prediction of Fetal
Chromosome Anomaly: Genetic computing

| Chromosome Anomaly | Area Under the ROC Curve |
|---|---|
| Down Syndrome* | 1.0 |
| Trisomy 18** | 1.0 |

*Phenyalanine, choline and Δ NT
**Glycerol, hydroxyvalerate and Δ NT

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above methods without departing from the scope of the invention, it is intended that all matter contained in the above description and shown in the accompanying drawing[s] shall be interpreted as illustrative and not in a limiting sense.

1. A method for determining a pregnant woman's risk of carrying a fetus with Down syndrome, the method comprising:
measuring concentrations of one or more metabolites selected from the group consisting of 2-hydroxy-butyrate, 3-hydroxy-isovalerate, acetamide, arginine, choline, glycerol, glycine, propylene glycol, carnitine, creatinine, phenylalanine and leucine in the pregnant woman's bodily fluid;
comparing the pregnant woman's one or more metabolite concentrations to concentrations of corresponding one or more metabolites obtained from pregnant women carrying Down syndrome fetuses and to concentrations of corresponding one or more metabolites obtained from pregnant women carrying chromosomally normal fetuses, wherein all of the metabolite concentrations are measured at same gestational age; and
predicting the pregnant woman's risk of carrying a fetus with Down syndrome, wherein a statistically significant change in the concentration of the one or more metabolites between the pregnant woman and the corresponding one or more metabolites from the pregnant women carrying chromosomally normal fetuses indicates a greater probability of carrying a fetus with Down syndrome.

2. The method of claim 1, wherein predicting the pregnant woman's risk of carrying a fetus with Down syndrome is also based on age of the pregnant woman, wherein increased age of the pregnant woman and the statistically significant change in the concentration of the one or more metabolites indicates the greater probability of carrying a fetus with Down syndrome.

3. The method of claim 1, further comprising the steps of:
measuring nuchal translucency of the pregnant woman's fetus; and
comparing the nuchal translucency of the pregnant woman's fetus to nuchal translucency of the chromosomally normal fetuses at same gestational age; wherein increased nuchal translucency of the pregnant woman's fetus as compared to the nuchal translucency of the chromosomally normal fetuses indicates greater probability of carrying a fetus with Down syndrome.

4. The method of claim 1, wherein the one or more metabolites are selected from the group consisting of propylene glycol, choline, carnitine, acetamide, phenylalanine and 2-hydroxy-butyrate; the group consisting of carnitine, acetamide and 2-hydroxy-butyrate; the group consisting of phenylalanine and choline; or creatinine.

5.-7. (canceled)

8. A method for determining a pregnant woman's risk of carrying a fetus with Trisomy 18, the method comprising:
measuring concentrations of one or more metabolites selected from the group consisting of 2-hydroxy-butyrate, acetate, choline, citrate, creatinine, ethanol, formate, glycerol, malonate, methanol, pyruvate, succinate, proline, hydroxy-isovalerate, hydroxy-valerate, and 3-hydroxy-butyrate in the pregnant woman's bodily fluid;
comparing the pregnant woman's one or more metabolite concentrations to concentrations of corresponding one or more metabolites obtained from pregnant women carrying fetuses with Trisomy 18 and to concentrations of corresponding one or more metabolites obtained from pregnant women carrying chromosomally normal fetuses, wherein all metabolite concentrations are measured at the same gestational age; and
predicting the pregnant woman's risk of carrying a fetus with Trisomy 18, wherein a statistically significant change in the concentration of one or more metabolites between the pregnant woman and the corresponding one or more metabolites from the pregnant women carrying chromosomally normal fetuses indicates a greater probability of carrying a fetus with Trisomy 18.

9. The method of claim 8, wherein predicting the pregnant woman's risk of carrying a fetus with Trisomy 18 is also based on age of the pregnant woman, wherein increased age of the pregnant woman and the statistically significant change in the concentration of the one or more metabolites obtained from the pregnant woman as compared to the concentration of the one or more metabolites obtained from pregnant women carrying chromosomally normal fetuses indicates the greater probability of carrying a fetus with Trisomy 18.

10. The method of claim 8, further comprising the steps of:
measuring nuchal translucency of the pregnant woman's fetus; and
comparing the nuchal translucency of the pregnant woman's fetus to nuchal translucency of the chromosomally normal fetuses at same gestational age; wherein increased nuchal translucency of the pregnant woman's fetus as compared to the nuchal translucency of the chromosomally normal fetuses indicates greater probability of carrying a fetus with Trisomy 18.

11. The method of claim 8, wherein the one or more metabolites are selected from the group consisting of glycerol, proline, hydroxy-isovalerate, 3-hydroxy-butyrate, and pyruvate; the group consisting of glycerol and hydroxyvalerate; the group consisting of 2-hydroxy-butyrate, creatinine and ethanol; or choline.

12.-14. (canceled)

15. The method of claim 1, wherein the bodily fluid is blood or urine.

16. The method of claim 15, wherein the bodily fluid is blood or a dried blood sample.

17. (canceled)

18. The method of claim 15, wherein the bodily fluid is urine.

19. The method of claim 1, wherein measuring the concentrations of the one or more metabolites is performed at a gestational age from 8 weeks to 18 weeks.

20. The method of claim 19, wherein measuring the concentrations of the one or more metabolites is performed at the gestational age from 9 weeks to 14 weeks.

21. The method of claim 20, wherein measuring the concentrations of the one or more metabolites is performed at the gestational age from 10 weeks to 13 weeks.

22. The method of claim 3, wherein the nuchal translucency is measured at a gestational age from 9 weeks to 14 weeks.

23. The method of claim 22, wherein the nuchal translucency is measured at the gestational age from 10 weeks to 13 weeks.

24. The method of claim 1, further comprising following the step of predicting the pregnant woman's risk of carrying a fetus with Down syndrome, comparing said pregnant woman's risk with a selected risk cut-off value to determine whether further testing is needed, wherein the further testing is needed if the pregnant woman's risk is greater than the selected risk cut-off value.

25. The method of claim 8, further comprising following the step of predicting the pregnant woman's risk of carrying a fetus with Trisomy 18, comparing said pregnant woman's risk with a selected risk cut-off value to determine whether further testing is needed, wherein the further testing is needed if the pregnant woman's risk is greater than the selected risk cut-off value.

26. The method of claim 24, wherein the further testing is selected from the group consisting of amniocentesis and chorionic villus sampling (CVS).

27. A computer-readable medium having stored thereon an array of normalized metabolite concentration values and a program of instructions executable by a processor to compare a pregnant woman's bodily fluid sample metabolite concentration value to a corresponding normalized metabolite concentration value obtained from pregnant women carrying chromosomally normal fetuses to predict the pregnant woman's risk of carrying a fetus with Down syndrome or a fetus with Trisomy 18.

28.-50. (canceled)
